(19) United States
(12) Patent Application Publication
(10) Pub. No.: US 2007/0111257 A1
(43) Pub. Date: May 17, 2007

(54) IMPROVED PROTEIN EXPRESSION COMPARISON ASSAY RESULTS AND APPLICATIONS

(76) Inventor: David E. Kohne, San Diego, CA (US)

Correspondence Address: WESLEY B. AMES, 7031 LOS VIENTOS SERENOS, ESCONDIDO, CA, 92029, US

(21) Appl. No.: 11/456,170

(22) Filed: Jul. 7, 2006

Related U.S. Application Data
(60) Provisional application No. 60/697,118, filed on Jul. 7, 2005.

(51) Int. Cl. G01N 33/53; G06F 19/00

(52) U.S. Cl. 435/7.1; 702/19

(57) ABSTRACT

The invention proves a method and means to produce protein expression comparison assay results and for using the improved protein expression comparison results for producing improved results for any other application which utilizes said improved protein expression results.

### RELATED APPLICATIONS

This application claims the benefit of Kohne, U.S. Provisional Appl. 60/697,118, filed Jul. 7, 2005, which is incorporated herein by reference in its entirety.

### FIELD OF THE INVENTION

The present invention relates to methods for obtaining improved results from protein expression assays and uses of such improved results.

### BACKGROUND OF THE INVENTION

The following discussion is provided solely to assist the understanding of the reader, and does not constitute an admission that any of the information discussed or references cited constitute prior art to the present invention.

The number of protein molecules of a particular type per cell is often termed the protein abundance for that protein. Different proteins are typically present in a cell or sample in different abundances. The same protein type is often present in different abundances in different cells of the same and different types. A protein which has a higher abundance in cell 1 than in cell 2 is said to be up-regulated in cell 1 and/or down-regulated in cell 2. A protein which has the same abundance in cell 1 and cell 2 is said to be unregulated. The ratio of (the particular protein abundance in cell 1÷the abundance of the same protein in cell 2) is termed the Differential Protein Expression Ratio, or the DPER. The True DPER or T-DPER value, is the DPER value which actually exists for the particular protein in the compared cell samples.

Prior art often experimentally determines a quantitative DPER value for one or more particular proteins in compared cell samples of the same or different cell or tissue or organ types (3-10). Prior art practices and believes that such a prior art determined DPER value is equal to the T-DPER for the particular protein in the compared cell samples.

It is known that different cells of the same types, and different cells of different types, commonly contain different amounts of total protein and different amounts of different particular proteins. It is further known that differences in growth rates, cell stage cycle, cell size, cell ploidy, and nutritional states can cause compared cells to have different total protein contents/cell and differences in the amount and type of particular proteins present. Prior art rarely, if ever, determines the total protein/cell content of the compared cells, or the total protein/cell content for a particular category or groups of categories of proteins in the compared cell samples.

Prior art determination of the DPER value for one or more proteins for a cell sample comparison almost always compares equal amounts of compared cell sample protein preparations of interest. Such a compared protein preparation of interest category includes, but is not limited to, nuclear proteins, microsomal proteins, cytoplasmic proteins, total cellular protein, mitochondrial protein, ribosomal protein, chromosomal protein, and others. The almost universal prior art practice of comparing equal amounts of protein from the compared cell samples of interest is termed the Equal Addition Rule, or the EA Rule.

For the prior art belief and practice that the prior art produced particular protein DPER values are biologically correct to be valid, the prior art assay relationship (the compared particular protein ACR value in the assay)=(the particular protein T-DPER value for the assay), must be true. Here (the compared particular protein ACR value)=(the number of sample 1 particular protein molecules in the assay comparison)÷(the number of same particular protein molecules from sample 2 compared in the assay). Prior art believes and practices that this (ACR=T-DPER) relationship is true. Since prior art believes and practices that a prior art assay measured particular protein DPER value is biologically correct, within the accuracy of the assay, then prior art further believes that (the assay measured and normalized particular protein DPER value)=(particular protein ACR value)=(particular protein T-DPER value).

Prior art gene expression analysis studies often assume that for different compared cell samples certain particular genes are present at the same RNA copy per cell numbers or abundance, i.e. a particular gene (PG) RNA in one cell sample has the same abundance as the same PG in the compared cell sample. Such PG RNAs are termed Housekeeping Genes (HG). Prior art assumes this in order to facilitate the normalization of gene expression assay results. The uses of naturally occurring HG RNAs for facilitating the normalization of PG RNA expression analysis results is discussed extensively in U.S. Provisional Patent application 60/687,526 and U.S. patent application Ser. No. 11/421,961, both of which are incorporated in their entireties herein. Naturally occurring PG RNAs have not been validly demonstrated to occur.

Similarly, it has not been validly demonstrated that one or more certain particular proteins (PP) or HG PPs are naturally present at the same copy per cell or abundance in all or many compared cell samples analysed in protein expression analysis assays. Natural and artificial HG RNAs and their characteristics and uses and methods for facilitating and improving the normalization of cell sample particular gene (PG) expression analysis results are discussed extensively in U.S. provisional Patent Application U.S. 60/687,526 and U.S. patent application Ser. No. 11/421,961. These discussions are directly applicable to the use of AHGPs for facilitating and improving the normalization of cell sample PP expression results and are incorporated in their entireties herein.

### SUMMARY OF THE INVENTION

The present invention addresses the problems of obtaining reliable and accurate assay results for protein expression assays, protein expression profiles, and the like. Current conventional methods produce results and expression profiles which are known to be incorrect or cannot be known to be correct. In many cases, the errors or potential errors are of significant magnitude that the accuracy and/or interpretability of the assay or application involving the assay are compromised and can result in incorrect interpretations and inferences. This invention improves such results, interpretations, and inferences by providing assay results which are improved due to improved normalization, such that the accuracy, interpretability, and/or additional properties of the results and applications are improved.

Thus, in a first aspect the invention concerns a method for producing improved particular protein (PP) expression analysis assay results (IR) for at least one cell sample. The method involves determining the number of cells or cell equivalents which are analyzed in the assay for each analyzed cell sample; and normalizing the assay measured PP expression comparison results for the number of cell sample cells analyzed in the assay, where the normalization produces assay results which are known to be improved in normalization and interpretability relative to such cell sample PP expression assay results obtained by prior art normalization practice.

Another aspect concerns a method for identifying a particular cell sample type of interest, and involves comparing protein expression profiles (PEPs) which incorporate improved results of at least one cell sample type of interest and at least one reference cell sample type of interest; and identifying the cell sample type of interest based on best match comparison of the respective PEPs. Such identification can be performed for a plurality of different cell types using one or a plurality of PEPs.

A further aspect concerns a method for identifying a set of particular proteins (PPs) which may be used to identify or characterize a particular cell sample type of interest, where the method includes determining improved PP expression results for a plurality of PPs in the particular cell sample type of interest and in at least one reference cell sample type; and identifying, and optionally further analyzing, PPs which are differentially expressed in the cell sample of interest compared to said reference cell sample type. The method may also include selecting at least a subset of the differentially expressed PPs as the set of PPs (i.e., a discrimination set) which may be used to identify or characterize said cell sample type of interest. In many embodiments, the improved PP expression results are compiled as a PEP for one or more cell samples of the said cell sample type of interest and/or for one or more cell samples of a specified reference cell sample type of interest; and the identifying PPs which are differentially expressed involves comparing protein expression profiles (PEPs) of at least one cell sample type of interest and at least one reference cell sample type of interest.

In embodiments in which a set or subset of particular proteins is selected as a discrimination set, the selecting can involve identifying from a set of differentially expressed PPs a discrimination set of one or more PPs which can be used to reliably, selectively, and specifically identify individual cell samples of the type of interest and to distinguish said cell samples of interest from the specific reference cell sample type. The bases for such selecting can include, for example, the magnitude of the differential expression for a PP, the consistency of occurrence and direction of the differential expression for a PP, the magnitude and the consistency of occurrence and direction of the differential expression for a PP.

In certain embodiments, the selecting involves application of one or more of the following methods: a linear discriminant method, a K-nearest neighbor method, a neural network method, a decision tree method, a partially supervised method, a class discovery method, a hierarchical agglomerative clustering method, a hierarchical divisive clustering method, a non-hierarchical K-means method, a self organizing maps and trees method, a principal component analysis method, a relationship between clustering and a principal component method, a protein shaving method, a clustering in discretised space method, a graph based clustering method, a Bayesian model method, a fuzzy clustering method, a clustering of proteins and samples method, a data mining analysis method, a systems biology analysis method, an independent component analysis method, and a direct comparison method.

A related aspect concerns an improved set of cell sample type discrimination particular protein (PP) molecules which includes a set of PP molecules which provide specific detection of individual PPs identified by the method of an embodiment of the preceding aspect which reliably, selectively, and specifically identify individual cell samples of the type of interest, or distinguish said cell sample type of interest from at least one specific reference cell sample type, or both, based on improved PP expression results.

In particular embodiments, the molecules are labeled or unlabeled or both; the set of discrimination molecules includes a set of capture protein molecules, the set of discrimination molecules includes a protein microarray; the set of discrimination molecules provides identification of cells of a cancer, cells infected by an infectious agent, cells of a developmental state, cells responsive to a bioactive molecule, and/or cells exposed to a defined environmental condition.

Similarly, in a closely related aspect and/or in embodiments of the preceding aspect, the invention concerns a set of a set of identifier reagents which can be used to detect and/or identify the discrimination set molecules. Such identifier reagents may be of various types, such as antibodies or antibody fragments, aptamers, nucleic acids, specific ligands or ligand analogs, and specific substrates. Thus, in particular embodiments, the set of identifier reagents includes a set of capture oligonucleotides molecules or a set of capture protein or antibody molecules, the set of identifier reagents includes an oligonucleotide microarray, and/or the set of identifier reagents includes a protein microarray.

Another aspect provides a method for identifying improved sets of PPs for an application utilizing PP expression results. The method includes obtaining improved PP expression results, e.g., using a method of the first aspect above, for at least one application pertinent PP; and selecting a discrimination PP set (and optionally corresponding identifier reagents) based on differential PP expression of said PP in at least one application pertinent cell sample type. An application pertinent cell type can, for example, be identified based on a cellular process associated with said application which is present at a desired level (which may include being absent) in the particular cell type.

In particular embodiments, the discrimination PP set is selected utilizing the method of an aspect described above for identifying a set of particular proteins (PPs) which may be used to identify or characterize a particular cell sample type of interest.

In certain embodiments, the application includes one or more of: a data mining analysis, a systems biology analysis, a regulatory pathway identification, or analysis, or monitoring, or any two, or all three, a drug or bioactive compound or biomarker discovery and identification, a drug or bioactive compound or biomarker validation, a drug or bioactive compound or biomarker development, a drug or bioactive compound efficacy analysis, a drug or bioactive compound safety evaluation, a drug or bioactive compound toxicity evaluation, a drug or bioactive compound QA/QC evaluation, a drug or bioactive compound manufacturing monitoring, a drug or bioactive compound or biomarker related diagnostic test development or use or both, a particular cell sample of interest related diagnostic test development or use or both, a disease or pathologic state or both detection or evaluation or both, a disease or pathologic state or both detection or evaluation or both, before and after administration of a therapeutic treatment, a disease or pathologic state or both detection or evaluation or both before and after drug administration, a disease or pathologic state or both detection, monitoring, or prognosis evaluation or any two or all three, a disease or pathologic state or both detection, monitoring, or prognosis evaluation or any two or all three, before or after drug or other treatment or both, a drug or bioactive compound commercial product candidate selection, a drug or bioactive molecule related clinical trial monitoring, a drug or bioactive compound commercial product candidate market segment identification, a drug or bioactive compound effectiveness and safety in the treated patient evaluation, a drug or bioactive compound prescription to the patient selection, and a monitoring of drug or biomolecule effectiveness or toxicity or both in the treated patient, wherein said monitoring may be long or short term or both.

In particular embodiments, the method also includes providing a set of particular protein (PP) identifier reagents, where members of the set of identifier reagents provide specific detection of corresponding members of a discrimination PP set. For example, the set may include at least 2, 3, 4, 5, 7, 10, 15, 20, 30, 50, 100, 200, 300, 400, 500, 700, or 1000 such identifier reagents. Such identifier reagents may, for example, be antibodies or antibody fragment molecules, specific binding ligands or ligand analogs, substrate or substrate analog molecules, and the like.

Likewise, a related aspect concerns a method for producing improved results for an application which directly or indirectly utilizes protein expression results and/or at least one protein expression profile (PEP) for at least one PP, which involves utilizing at least one improved PEP directly or indirectly in the application, thereby producing improved application results. The results and/or PEPs may, for example, be produced by a method as described above.

In particular embodiments, the PEP is a particular cell sample or cell sample type PEP; the PEP includes a cell sample PEP which includes a set of one or more regulated PPs which may be used to selectively and specifically identify a particular cell sample type or a particular cell sample type physiological state (PS) of interest or both; the PEP includes a cell sample PEP which includes a set of one or more regulated PPs which can be used to selectively and specifically identify a particular cell sample type or physiological state of interest.

The invention provides in another aspect an improved method for identifying regulated PPs which are regulated in response to exposure to a particular treatment by comparing at least one improved PP expression profile (PEP) incorporating improved results for at least one cell sample exposed to the treatment with at least one improved PEP for at least one reference cell sample, thereby identifying PPs with differential expression in the treated cell sample. The improved results and/or improved PEPs can be provided by a method as described above. The method can also include selecting PPs which identify and/or characterize exposure to the treatment.

In particular embodiments, cells in the treated cell sample are subjected to the treatment and cells of the reference cell sample are not subjected to the treatment; one or more selection processes are used to identify and rank the regulated PPs based on the magnitude and direction of the change in expression level for the PP in the treated cell sample; one or more further selection processes are used to evaluate the suitability of each of the regulated PPs for the purpose of the comparison, and the method includes interpreting and ranking and arranging the members of the set of regulated PPs and their characteristics in a manner which reflects their suitability of use for the purpose of the comparison and identification; the selection process involves application of one or more of the following analysis techniques or methods: a linear discriminant method, a K-nearest neighbor method, a neural network method, a decision tree method, a partially supervised method, a class discovery method, a hierarchical agglomerative clustering method, a hierarchical divisive clustering method, a non-hierarchical K-means method, a self organizing maps and trees method, a principal component analysis method, a relationship between clustering and a principal component method, a protein shaving method, a clustering in discretised space method, a graph based clustering method, a Bayesian model method, a fuzzy clustering method, a clustering of proteins and samples method, a data mining analysis method, a systems biology analysis method, an independent component analysis method, and a direct comparison method

In particular embodiments, the method includes exposing at least one of a plurality of matching cell samples or a portion of a cell sample to a treatment of interest thereby forming a treated cell sample, while at least one other of the cell samples or portions is not exposed to the treatment of interest, and constitutes the reference sample, and a method as described above may be used to produce a PEP for each of said cell samples.

In certain embodiments, the particular treatment includes one of or a combination of two or more of, the following treatments: exposure to a compound in a compound screening library, exposure to a pharmaceutical drug screening hit, exposure to a pharmaceutical drug lead, exposure to a pharmaceutical drug, exposure to a potentially toxic compound, exposure to a toxic compound, exposure to an illegal drug, exposure to protein binding compound, exposure to an infectious agent, exposure to a virus, exposure to a bacterium, exposure to radiation, exposure to light, exposure to ultraviolet light, exposure to a temperature shift, exposure to a biological stress condition, exposure to a psychological stress condition, exposure to a physical condition, exposure to a bioactive compound, and exposure to an environmental condition.

In particular embodiments, the selecting involves identifying from a set of differentially expressed PPs a discrimination set of one or more PPs which can be used to reliably, selectively, and specifically identify individual treated cell samples subjected to a treatment of interest and to distinguish the cell samples of interest from the specific reference cell sample; the bases for the selecting include the magnitude of the differential expression for a PP, the consistency of occurrence and direction of the differential expression for a particular PP, and/or the magnitude and the consistency of occurrence and direction of the differential expression for a particular PP; the selecting involves application of an analysis technique or method as indicated for aspects involving selection above.

In certain embodiments, the method includes providing a set of a set of PP identifier reagents, wherein members of said set of molecules provide specific detection of corresponding members of said discrimination PP set. Such identifier reagents may be as described for an aspect above and/or identified by a method as described above.

The invention also concerns providing improved higher order application results, that is, applications which indirectly utilize improved protein expression results. Thus, another aspect concerns a method for producing higher order application results which are improved in one or more of qualitative accuracy, quantitative accuracy, interpretability, reproducibility, intercomparability, and utility, relative to prior art produced higher order application results, where the method involves using any of the methods described herein for producing improved results and/or improved protein expression profiles to produce such improved results and or profiles, and utilizing one or more of those improved results and/or profiles directly or indirectly in a higher order application to produce higher order application results which are improved in one or more of qualitative accuracy, quantitative accuracy, interpretability, reproducibility, intercomparability, and utility, relative to prior art produced higher order application results.

Another aspect of the invention concerns a method for producing improved information and results concerning the physiological state of cells in a cell sample of a particular cell type of interest, and involves utilizing one or more particular physiological state PP expression profiles (PS PEPs) to identify the physiological state of different samples of the particular cell type of interest, where particular PS PEPs for the particular cell type of interest selectively distinguish a particular physiological state (PS) for the particular cell type of interest. The PS PEPs are improved by the incorporation of improved protein expression results and the information and results are improved in one or more of qualitative accuracy, quantitative accuracy, interpretability, reproducibility, intercomparability, and utility, relative to prior art produced information and results. The method may also include monitoring the physiological state and analyzing the monitoring results to evaluate and determine the physiological state of the particular cell type sample of interest over time and under changing or changed conditions.

In particular embodiments, a method as described above for producing the improved results and/or improved PEPs is utilized to produce one or more physiological state PP expression profiles (PS PEPs) for the particular cell type of interest which selectively distinguish a particular physiological state (PS) for said particular cell type of interest; the particular cell type is or includes: a eukaryotic cell type, a prokaryotic cell type, a plant cell type, a bacterial cell type, a pathogenic bacterial cell type, a yeast cell type, a fungal cell type, a mammalian cell type, a human cell type, an in vitro grown cell type, an immortalized cell line type, an in vivo grown cell type, an infectious organism or agent infected cell type, a virus infected cell type, a genetically modified cell type, and/or an in vivo or in vitro cell type used for producing or manufacturing a pharmaceutical agent or protein or small molecule or lipid.

In particular embodiments, the particular physiological state is or includes a state selected from the group consisting of: a cell cycle stage related PS, a cell growth state related PS, a cell size related PS, a differentiated state related PS, an undifferentiated state related PS, a toxic state related PS, a cell age related PS, an infectious state related PS, a nutritional state related PS, a drug or bioactive agent treatment of the cell type related PS, an environmental state related PS, a physical treatment of the cell type related PS, a psychological treatment of the cell type related PS, a chemical treatment of the cell type related PS, and a hormone treatment related PS.

Yet another aspect of the invention provides a method for producing improved clinical trial information and results which are improved in qualitative accuracy, quantitative accuracy, interpretability, reproducibility, intercomparability, and/or utility, relative to prior art produced such information and results, for the evaluation of one or more or all of the safety, dose, or efficacy of a drug or bioactive agent (BA). The method involves monitoring one or more improved PP expression profiles (PEPs) for drug or BA treated and untreated particular cell types of interest respectively for the appearance of one or more drug treatment desired effects or undesired effects or both in the treated cell types of interest, where the improved PEPs incorporate improved protein expression results. The method may also include analyzing the results of the monitoring to evaluate the safety, dose, and/or efficacy of the drug or BA treatment of the particular cell types of interest. The improved results and/or improved PEPs may be provided by a method as described above.

In particular embodiments, the particular cell type of interest is or includes at least one of the cell types described for other aspects herein; the PEP includes a complete PEP for the treated and untreated cell type or types of interest; the PEP includes a partial PEP specific for a particular treated or untreated cell type or types of interest; the PEP includes a combination of complete and partial PEPs for the treated or untreated cell type or types of interest; the desired or undesired effect includes the known desired effects of the drug or BA on the cell types of interest, the unknown potential desired effects of the drug or BA on the cell types of interest, the known undesired effects of the drug on the cell types of interest, and/or the unknown potential undesired effects on the cell types of interest.

The invention is beneficially applied in the context of patient care and determining effects of particular treatments, and the selection of beneficial treatments or avoidance of sub-optimal treatments. Thus, another aspect concerns a method for producing improved information and results concerning the efficacy and toxicity or both or the desired and undesired effects or both, of treatment for a patient being treated with a particular drug or bioactive agent (BA), or with a combination of a plurality of drugs or BAs or both, which is improved in one or more of qualitative accuracy, quantitative accuracy, interpretability, reproducibility, intercomparability, and utility, relative to such prior art produced information and results. The method involves monitoring one or more improved protein expression profiles (PEPs) of patient cell samples for drug or BA treated particular cell types of interest for the appearance of one or more drug treatment desired effects or undesired effects or both in said treated cell types of interest, wherein said improved PEPs incorporate improved protein expression results.

In particular embodiments, the method includes monitoring results to determine the effectiveness of the treatment or undesired effects of the treatment or both; the improved results and/or improved PEPs are provided by a method of an aspect above to produce cell type specific PEPs for the combination of the patient cell types of interest and drug or BA of interest; the method also includes comparing at least one PEP for a treated cell sample from the patient with at least one PEP for at least one untreated cell sample; the treated cell and/or the untreated cell sample is from the patient; a PEP includes a partial PEP specific for a particular treated or untreated cell type or types of interest; the PEP includes a combination of complete and partial PEPs for the treated or untreated cell type or types of interest; the desired or undesired effect is or includes the known desired effects of the drug or BA on the cell types of interest, the unknown potential desired effects of the drug or BA on the cell types of interest, the known undesired effects of the drug on the cell types of interest, the unknown potential undesired effects on the cell types of interest.

Another aspect of the invention concerns a method for producing improved patient bioactive agent treatment related health care, and involves utilizing the method of an embodiment of an aspect above for determining the effectiveness of the particular drug or bioactive agent (BA) treatment in a patient, and selecting a drug or BA treatment utilizing the determination of effectiveness information.

Similarly, the invention also concerns an aspect providing a method for producing improved patient drug or bioactive agent treatment related health care which involves selecting treatment for a patient based on comparison of improved protein expression results and/or at least one improved PEP for the patient, and at least one reference PEP indicative of patient response to the drug or bioactive agent treatment (or improved results for or more reference PPs). The improved results and/or improved PEPs can, for example, be produced by a method of an aspect described above for producing such improved results and/or improved PEPs.

In particular embodiments of the last two aspects, the patient suffers from a disease or condition for which the presence of certain allelic variants is indicative of variation in the effectiveness of treatment with said drug or bioactive agent or indicative of differences in effectiveness of different bioactive agents; the bioactive agent is a food, nutritional supplement, or nutritional compound.

In certain embodiments of the last two aspects, a method as indicated above is used for determining the effectiveness of the particular drug or bioactive agent (BA) treatment in a patient, and that effectiveness information is used to select a drug or BA treatment; the selecting includes continuation of treatment with said drug or bioactive agent; the selecting includes an increase in dosage of the drug or bioactive agent; the selecting includes a decrease in dosage of said drug or bioactive agent; the selecting comprises termination of treatment with said drug or bioactive agent; the selecting includes administration of an additional drug or bioactive agent; the effectiveness information includes information on the efficacy of the drug or bioactive agent in the patient; the effectiveness information includes information on the safety of the drug or bioactive agent in the patient; the effectiveness information includes tolerance of dosage level information in the patient.

An electronic representation of improved protein expression assay results and/or an improved PP expression profile (PEP), which includes electronic representations of a plurality of improved results obtained by the method of any of the aspects above for obtaining improved protein expression results and/or improved protein expression profiles.

In particular embodiments, the representation is on a computer display, or stored in a computer readable computer storage medium, e.g., volatile computer memory, a magnetic storage medium such as a computer hard drive or portable disk, an optical disk such as a CD or DVD or the like, or in a flash memory device, or the like.

A further aspect of the invention concerns a method for determining improved application results for an application which directly or directly utilizes improved protein expression assay results and/or improved protein expression profile (PEP) information, and involves entering data describing or derived from said PEP in computer accessible form, and operating on that data with a computer program comprising program steps to calculate the application results.

In particular embodiments, the data is in one or more computer accessible databases; the PEP includes data on expression of a plurality of particular proteins (e.g., a number as indicated for an aspect above); the data is stored in a computer readable medium, such as computer volatile memory (e.g., RAM), in a magnetic storage medium such as on a hard drive, an optical disc such as a CD or DVD or the like, a flash memory device, and the like.

In particular embodiments, the application is one indicated for an aspect above and/or the application includes an analytical technique as indicated for an aspect above.

In certain embodiments of each of the present aspects which involve obtaining and/or using improved protein expression results and/or protein expression profiles, the PP expression result includes a cell sample PP abundance value, i.e. the number of PP molecules per cell for a cell sample, or a PP differential protein expression ratio (PP-DPER) for a cell sample comparison, or both; the PP expression result includes a protein expression profile (PEP) for a cell sample consisting of one or more PP relative or absolute PP abundance results, or a differential protein expression profile(D-PEP) consisting of compared cell sample PEPs, or both.

In particular embodiments of each of the aspects of the present invention, the PP is an endogenous protein, an exogenous protein, a wild type protein, a mutant protein, an allelic variant protein; a splice variant protein, a human protein, a mammalian protein, a viral-encoded protein; a prokaryotic protein, a prion protein.

In yet another aspect, the invention concerns an improved protein array (e.g., a protein microarray chip), which includes a set of discriminator protein molecules or identifier reagents which are identified or selected by a method described above for such identification or selection.

A related aspect concerns an assay kit which includes at least one improved protein array as specified for the preceding aspect.

In particular embodiments, the kit also includes instructions for carrying out an assay using the array, or additional components for carrying out an assay using the array, or both, packaged with the array; the additional components include one or more of binding solution, wash solution, detection solution, and detection labeling molecule; at least one additional component solution is provided in dry form suitable for addition of water to form an aqueous solution.

Still another aspect of the invention concerns a method for normalizing protein expression results for at least one cell sample, by determining the assay SCR for a differential protein expression assay, and normalizing results of the assay for an assay SCR≠1. Such normalization can be performed for a plurality of particular proteins and/or cell samples.

Particular embodiments are as described herein for an aspect concerning methods for obtaining improved results.

Another aspect concerns a method for improving normalization of protein expression assay results and the uses thereof by using one or more different control PP molecules to artificially create the presence of one or more housekeeping gene (HG) PPs in each cell sample protein preparation analysed in a protein expression analysis assay. The use of artificial housekeeping gene particular proteins can also advantageously be used in embodiments of each of the aspects of the invention concerning obtaining or providing improved protein expression assay result. Herein such artificially created HG PPs are termed artificial housekeeping gene PPs or AHGPs They can particularly be utilized for the purpose of facilitating the normalization of the assay measured cell sample protein expression analysis assay results for the number of sample cells or sample cell equivalents analysed, as well as other assay variables. The method involves mixing a known amount or number of each of one or more AHGPs with a cell sample protein preparation to be analysed in the assay, for which the number of sample cells or sample cell equivalents present in the assay analysed cell sample protein preparation is known. This creates an assay analysed cell sample protein mixture which contains one or more different control PP AHGPs, each of which has a known abundance value in the analysed cell sample protein preparation. After measuring the assay PP expression results the AHGP results can be used to facilitate the normalization of the cell sample PP expression results for the number of sample cells analysed in the assay as well as other assay associated variables in order to produce invention improved cell sample PP expression results. In particular cases, at least 1, 2, 3, 4, 5, 6, 7, 10, or even more different AHGPs are used in a particular assay or a particular cell sample; different AHGPs are used in different cell samples in an assay run.

In certain embodiments of each of the aspects described for the invention, the cells in the cell sample can be of essentially any type, for example, normal cells; abnormal cells; untreated cells; treated cells; physically treated cells; chemically treated cells; drug treated cells; bioactive compound treated cells; cells from a psychologically treated individual; drug candidate treated cells; toxic compound treated cells; differentiated cells; undifferentiated cells; biological agent infected cells; virus infected cells; cells from an individual infected by a pathogenic bacterium; cells from an individual infected by a eukaryotic microbe; neoplastic cells; cancer cells; diseased cells; pathological cells; in vitro cultured cells; in vitro cultured cells of an immortalized cell line; in vivo sampled cells; in vivo sampled cells of a particular tissue; prokaryotic cells; eukaryotic cells; temporally treated cells; mammalian cells; mouse cells; rat cells; and human cells. Cells can further be of a particular age, development stage, and/or nutritional status.

In certain embodiments of each of the aspects of this invention in which protein expression results and/or improved PEPs are obtained and/or used, one or a plurality of cell samples can be analyzed, e.g., at least 2, 3, 5, 10, 20, 50, 100, 200, 300, 500, 1000, or even more or a number in a range of 1-5,2-10, 11-50, 51-100, 101-500, 501-1000, 1000-10000, among others.

In particular embodiments of each of the aspects of the invention in which improved results and/or improved PEPs are obtained and/or used, improved results (IR) for a plurality of different PPs are obtained and/or used, e.g., at least 2, 3, 5, 10, 20, 50, 100, 200, 300, 500, 1000, 10,000 or even more or a number in a range of 1-5,2-10, 11-50, 51-100, 101-500, 501-1000, 1000-10000, among others.

In certain embodiments of each of the aspects of this invention in which protein expression results and/or improved PEPs are obtained and/or used, PPs included in a cell sample PEP include at least one cellular protein, such as a regulatory protein, a membrane protein, a protein from an infectious biologic agent, a biomarker protein, a drug or bioactive compound candidate or target PP, and/or a pathologic- or disease-related protein.

In each of the present aspect involving generation or use of at least one PEP, the PEP is improved in quantitative accuracy, qualitative accuracy, or both, as compared to a PEP compiled from results which are not IRs; the PEP is improved in interpretability as compared to a PEP compiled from results which are not IRs; the PEP is improved in reproducibility as compared to a PEP compiled from results which are not IRs; the PEP is improved in intercomparability as compared to a PEP compiled from results which are not IRs; the PEP is improved in utility as compared to a PEP compiled from results which are not IRs. For each of the preceding aspects, such improvement can also be obtained for an application in which the PEP is directly or indirectly used.

In each of the present aspects involving determination or use of improved results or PEPs, the determining one or more PP improved results (IR) for said cell sample is performed using a protein or aptamer oligonucleotide microarray assay, a 2D gel electrophoresis assay, at least one affinity binding media method, a mass spectroscopy method, an immunoassay method, and/or an ELISA method.

In particular embodiments of each of the present aspects which involve determining or using protein expression profiles, improved results are obtained by using an embodiment of the first aspect above; the PEPs include expression results for a plurality of PPs, e.g., at least 2, 3, 5, 10, 20, 50, 100, 200, 300, 500, 1000, 10,000 or even more or a number in a range of 1-5, 2-10, 11-50, 51-100, 101-500, 501-1000, 1000-10000, among others; for a plurality of PPs, those PPs which are differentially expressed in the cell sample of interest are identified; improved results are obtained according to an embodiment of the first aspect above for one or more cell samples of one more cell sample types of interest and/or for or more cell samples of one or more reference cell sample types of interest, and the results are incorporated in at least one PEP; one or more one or more regulated PPs are detectably expressed in both the cell sample of interest and a reference cell sample type; one or more down-regulated PPs is not detectable as being expressed in one of the cell samples; the cell sample type of interest includes a plurality of separate different cell sample types; the reference cell sample type includes a plurality of separate different cell sample types; the at least one cell sample type of interest or said reference cell sample type or both includes a plurality of different cell sample types, e.g., at least 2, 3, 4, 5, 7, 10, 12, 15, 20, or more, or in a range specified by taking any two of these listed values as inclusive endpoints of the range.

In particular embodiments of aspects described above which include a direct or indirect application (which may be a higher order application) of the improved protein expression results and/or protein expression profiles, the application includes use of one or more of the following analysis techniques or methods: a linear discriminant method, a K-nearest neighbor method, a neural network method, a decision tree method, a partially supervised method, a class discovery method, a hierarchical agglomerative clustering method, a hierarchical divisive clustering method, a non-hierarchical K-means method, a self organizing maps and trees method, a principal component analysis method, a relationship between clustering and a principal component method, a protein shaving method, a clustering in discretised space method, a graph based clustering method, a Bayesian model method, a fuzzy clustering method, a clustering of proteins and samples method, a data mining analysis method, a systems biology analysis method, an independent component analysis method, and a direct comparison method.

Similarly, in particular embodiments of aspects described above which include a direct or indirect application of the improved protein expression results and/or protein expression profiles, the application is or involves one or more of: a data mining analysis, a systems biology analysis, a regulatory pathway identification, or analysis, or monitoring, or any two, or all three, a drug or bioactive compound or biomarker discovery and identification, a drug or bioactive compound or biomarker validation, a drug or bioactive compound or biomarker development, a drug or bioactive compound efficacy analysis, a drug or bioactive compound safety evaluation, a drug or bioactive compound toxicity evaluation, a drug or bioactive compound QA/QC evaluation, a drug or bioactive compound manufacturing monitoring, a drug or bioactive compound or biomarker related diagnostic test development or use or both, a particular cell sample of interest related diagnostic test development or use or both, a disease or pathologic state or both detection or evaluation or both, a disease or pathologic state or both detection or evaluation or both, before and after administration of a therapeutic treatment, a disease or pathologic state or both detection or evaluation or both before and after drug administration, a disease or pathologic state or both detection, monitoring, or prognosis evaluation or any two or all three, a disease or pathologic state or both detection, monitoring, or prognosis evaluation or any two or all three, before or after drug or other treatment or both, a drug or bioactive compound commercial product candidate selection, a drug or bioactive molecule related clinical trial monitoring, a drug or bioactive compound commercial product candidate market segment identification, a drug or bioactive compound effectiveness and safety in the treated patient evaluation, a drug or bioactive compound prescription to the patient selection, and a monitoring of drug or biomolecule effectiveness or toxicity or both in the treated patient, wherein said monitoring may be long or short term or both.

Additional embodiments will be apparent from the Detailed Description and from the claims.

### DETAILED DESCRIPTION OF THE PREFERRED EMBODIMENTS

Definitions of Selected Terms

Preliminarily, the meanings of selected terms used herein will be provided. The meanings of other terms not expressly stated herein will be the meaning as used in U.S. patent application Ser No. 11/421,961, and if not indicated herein or in that application, then the meaning in the context it is used as understood by persons of skill in the art.

In the context of protein expression, the term “abundance” refers to the number of protein molecules per cell for a particular protein.

In the context of a particular application, the term “application pertinent cell sample type” refers to a cell sample type which is selected to have at least one property which is pertinent to the application, e.g., cells which exhibit a detectable response to a particular change in conditions or cells from an organism which is the intended target of a particular treatment.

The term “biomarker protein” is used to refer to a protein which serves or can serve as a marker for a particular normal or abnormal property of a cell or organism, such as response to a drug treatment, a bioactive agent treatment, and/or an environment treatment, or to exposure to or infection by an infectious agent, or as a marker for particular or general physiological states, e.g., normal, abnormal, or pathological. Biomarkers are sometimes referred to as “surrogate markers”.

As used herein in reference to assays and assay kits and systems, the term “commercial” indicates that the kit, etc, is available for sale generally to individuals and/or business entities (e.g., profit and non-profit business entities). In contrast, the term “homebrew” indicates that the kit is not available for general sale. Typically such homebrew assays and materials are adapted for use by a particular laboratory and are not distributed beyond the particular business entity and/or collaborators.

The term “DPE” refers to Differential Protein Expression, which generally refers to the concept that the same particular protein can be expressed to a different extent in different cells. In addition, different particular protein in different cells (DPDS), and different particular genes in the same cell (DPSS), can also be differentially expressed. Such a difference in gene expression between compared particular genes is generally described in terms of a DPE ratio or DPER. A DPER value which has been normalized for one or more assay variables is termed a N-DPER. The biologically accurate DPER value for a cell sample comparison is termed the true DPER or T-DPER.

The term “set of cell sample type discrimination molecules” and like terms refer to a set of one or more expressed PPs which are expressed in a cell sample type of interest, and which are specific for or characteristic of the cell sample type of interest and which can be used to identify or detect the presence of that cell sample type and/or to discriminate that cell sample type from at least some and preferably all other cell sample types which may be present in a particular cell sample.

The term “particular protein (PP) identifier reagent means a protein, nucleic acid, or other chemical molecule, complex, or set of molecules which can be used to specifically detect and identify a PP molecule, especially a cell sample type discrimination PP molecule. Usually such identifier reagents will be identified and/or provided as component of a set of such reagents which can be used to specifically detect one or more such PP molecules.

The phrase “identify or characterize a particular cell sample type of interest” means respectively to determine the type of the cells in the cell sample, or to determine one or more properties of the cells in the cell sample.

As used in the context of the present invention, the terms “improved”, “improved results”, “improved assay” and like terms indicate that the reference item(s) or process has at least one better or more advantageous characteristic such that the item as a whole is better, more advantageous for a use, or otherwise preferred. Such improvement is commonly better in normalization, completeness of normalization, accuracy, reproducibility, interpretability, validity, and/or reliability and utility. Improvements in normalization are generally obtained according to the invention described herein by validly and/or more completely normalizing for pertinent UNFs and CNFs which were not previously completely and/or validly normalized for. Improvements in reliability may, for example, mean that the validity of the value, result, or process which was previously invalid or of uncertain validity have increased validity, e.g., either shown to be valid or correct, or the risk of invalidity or incorrect results or interpretations has been reduced. For example, the probability that a particular normalization factor or process is invalid may be reduced, even if not eliminated.

In the present context when used to refer to assay results, the phrase “known to be improved” means that the process of obtaining the results is based on normalization procedures which are known or shown to be valid or at least to be more likely to be valid than results produced using prior normalization procedures. Such procedures are distinguished, for example, from normalization procedures which are not known or shown to be valid (e.g., because they are based on assumptions which are themselves of unknown validity) or which are known or shown to be invalid (e.g., because they are based on assumptions which are known or shown to be invalid).

Unless clearly indicated to the contrary (e.g., clearly limited to natural or unmodified molecules), the terms “nucleic acids” and “nucleic acid molecules” refer to molecules which are made of covalently linked chains of nucleotides and/or nucleotide analogs, and thus includes unmodified nucleic acid molecules, modified nucleic acid molecules, and analogs of nucleic acid molecules. The terms further include oligonucleotides as well as longer such chains, including without limitation, siRNAs, miRNS, and full-length mRNAs, cDNAs, and cRNAs.

Similarly, unless clearly indicated to the contrary, the term “oligonucleotide” is used to refer to relatively short nucleic acid molecules, that is molecules up to 200 linked nucleotides and/or nucleotide analogs. Such oligonucleotides may also be referred to as oligos or oligomers. Longer nucleic acid molecules may be referred to as polynucleotides, or simply nucleic acids or nucleic acid molecules.

As used herein in connection with normalization of protein expression assays, the phrase “prior art normalization process” and like terms refer to a normalization process or method which has previously been used in connection with protein expression assays which does not incorporate the present improved normalization methods.

The phrase, “prior art normalization process which relies on the usual necessary prior art assumptions for validity” and phrases of like import refers to a normalization process commonly utilized by the prior art which relies on the validity of one or more necessary assumptions for its validity.

As used herein, the term “protein expression profile” (PEP) refers to an indication of the expression extent or abundance values for a part, or all, of the proteins or polypeptides present in a sample population. Thus, a protein expression profile for population analyzed should indicate the proteins which are detectable as expressed and those which are not detectable as expressed, and can provide a quantitative measure of the extent, either absolute or relative, of expression for one or more proteins. The expression profiles of two or more sample protein populations can be compared to identify differences in expression extents, which exist between the different samples. A Differential Protein Expression (DPE) profile resulting from the comparison of two different individual protein expression profiles, should indicate whether a protein is expressed in both cell samples, and can provide a quantitative measure, either absolute or relative, of a protein's number of molecules per cell which is present in each sample. Thus, a protein expression profile is similar to a gene expression profile.

In the context of comparisons between values (e.g., total protein content per cell, or total number of a particular protein or class of particular protein per cell), unless otherwise specified, the term “significantly” indicates that the values differ to a statistically significant extent which is also substantial in the context of the particular assay. Further, specifically in the context of differences in total protein content per cell, or total number of a particular protein per cell, indication that such difference is “not primarily due” to a specified cause or condition means that the specified cause or condition is responsible for less than ½ of the magnitude of the difference. In this same context, the phrase “expressed only in the compared sample which is associated with the larger measured value” means that the particular gene(s) are not expressed or not detectably expressed in cells from one of the two compared samples and are substantially and meaningfully expressed in cells of the other compared sample. Thus, it does not necessarily mean that there was absolutely no expression in the one set of cells, it only means that the expression in one set was insignificant compared to the expression level in the other.

A “PP expression extent per cell” or “PP cell sample expression extent per cell” refers to a quantitative measure of the PP expression extent which is measured in terms of some parameter other than the actual number of PP molecules. One exemplary measure which can be used is the number of fluorescence units associated with the PP in the cell sample assay results. In this case, the PP expression extent per cell would be described in terms of PP fluorescent units per cell, which can be converted to PP molecules per cell if the number of fluor units per PP molecule for the assay is known or determined.

In connection with protein expression assays and protein expression comparisons, the term “SC) refers to the sample cell number, which is equal to the number of a cell sample's protein cell equivalents (CE) which are analyzed in the assay. For a cell sample comparison, the “SCR” is equal to the ratio of the compared cell sample SC values.

### DESCRIPTION

The invention described herein provides methods and means to obtain particular protein DPER value results which are improved, relative to prior art practice produced particular protein DPER results. The practice of the invention provides particular protein DPER results which, as a result of being known to be improved in normalization relative to prior art practice produced particular protein DPER results, are improved with regard to quantitation and/or biological accuracy and/or interpretability and/or intercomparability and/or utility, relative to prior art produced particular protein DPER results. The practice of the invention is necessary in order to obtain particular protein DPER values which can be known to be biologically correct.

Because of the improved nature of such particular protein DPER results, the invention provides methods and means for obtaining improved global proteome or particular protein expression profiles and/or improved proteome or particular protein subset protein expression profiles for one or more sets of cell sample or tissue sample comparisons. Here, the proteome can be the total protein content of a cell or cell sample or the protein content of a subset of category of the cell or cell sample total proteome. The invention also provides means and methods for obtaining improved data mining and systems biology analysis results from the intercomparison, correlation, and the analysis, of improved particular protein DPER results and the improved proteome DPER profiles. Further, the invention provides methods and means for producing improved results from any process or application which utilizes particular protein DPER results.

The invention has application to all methods for particular protein comparison, including the comparison of, the same particular protein in different cell samples (SPDS), and different particular proteins in different samples (DPDS). Such methods and means are broadly applicable to all kinds of cell sample or tissue sample particular protein comparison assays or analyses. Such methods and means can be used to produce improved particular protein DPER results for cell ample and tissue comparisons of all kinds, which include, but are not limited to, the following. (a) Normal cells or tissues of all kinds and ages. (b) Differentiated cells and tissues of all kinds and ages. (c) Cells and tissues of all kinds in different cell cycle, growth, or metabolic states of all kinds. (d) Cells and/or tissues and/or organisms of all kinds associated with pathogenic or non-pathogenic viruses, cells, or organisms, of all kinds. (e) Cells and/or tissues and/or organisms of all kinds which are associated with a non-genetic or genetic disease state of any kind. (f) Cells and/or tissues and/or organisms of all kinds associated with a genetic change of any kind, whether created by man or nature. (g) Cell and/or tissues and/or organisms associated with or treated with bioactive, drug, toxic, non-toxic, mutagenic, inhibitor, or nutrient compounds, of all kinds, or any other chemical compounds, or combinations of such compounds. (h) Cells and/or tissues and/or organisms of all kinds associated with non-chemical treatments of all kinds such as radiation, temperature, mechanical, and stresses of all kinds. (i) Cultured cells of all kinds associated with substances or conditions which can affect cell growth rates, cell cycle stage, the cell cycle distribution profile, cell size, cell recombinant and other protein production capability, cell adherence to surface, cell morphology, cell differentiation, and other cell characteristics. Such substances and conditions include but are not limited to pCO₂, pO₂, pH, stir rates and shear forces, osmotic pressure, redox potential carbohydrate levels, growth factors, steroids and other hormones, lipids and fatty acids, amino acid levels, eicosanoids and eicosanoids precursors, cations, anions, cytokines, vitamins, nucleic acid precursors, and others.

Thus, as indicated above, the invention has application in essentially all prior art methods for quantitatively determining the absolute and/or relative extents of expression of a particular protein (PP) in a cell sample comparison. These methods include but are not limited to protein specific or protein activity specific arrays or microarrays, protein specific immunoassays or aptamer or affinity assays or ELISA methods, as well as 2D gel electrophoresis and liquid chromatography and mass spectrometry assays. and combinations of these methods and others (1-5). The invention relates to the incorporation of various modes of practice of the invention into such methods for comparing the quantitative absolute and/or relative extent of expression of particular proteins in compared cell samples and determining quantitative measures for the differential protein expression.

The invention also applies to all applications which utilize one form or another of the assay-measured differential protein expression results. Such assay results include, but are not limited to, particular protein differential expression results, global and non-global protein expression profile results, proteome differential protein expression profiles, protein expression data mining results, and systems biology results, as well as data mining and systems biology analysis results which utilize both gene expression and protein expression results. Said applications include, but are not limited to, all biological organisms such as eukaryotes, prokaryotes, viruses, and therefore microbes, plants, and animals of all kinds. The invention relates broadly to biological research and development of virtually all kinds, and to medical, agricultural, environmental, industrial, and manufacturing, applied, and service, applications, which are related to biology.

More specifically the invention is applicable to virtually all areas of biological research and development which include but are not limited to, physiology, genetics and gene regulation, epidemiology, evolution, ecology, endocrinology, immunology, nutrition, toxicology, oncology and cancers of all kinds, stem cell studies related to embryogenesis and differentiation, organ and tissue and cell in vitro studies of all kinds, organ and tissue and cell transplantation of all kinds, virology, and microbiology, pathogenesis of all kinds, diseases of all kinds, and products and services which are associated with biological research and development.

The invention is further applicable to a large number of agricultural-related applications. These include, but are not limited to, the following. Essentially all areas of basic, applied, and industrial agricultural research and development, including the just described biological research and development areas. The areas of developing naturally and genetically improved plants and animals and bacteria for food production and other purposes. The areas of plant and animal diseases of all kinds, and disease mechanisms, and host-pathogen interactions. The areas of the discovery, development, validation, production, and use, of plant and animal antiviral agents, antimicrobial agents, antifungal agents, pesticides, plant and animal growth agents, and agricultural pharmaceutical agents of all kinds. The areas of agricultural ecology and toxicology. Products and services which are associated with the above-described areas of application.

The invention further relates to a large number of medical, both human and veterinary, related applications. These include, but are not limited to, the following. Essentially all areas of basic, applied, and industrial, medical research and development, including the above-described biological research and development areas. The pathogenesis, prevention, diagnosis, treatment, and cure of: infectious and non-infectious diseases of all kinds; genetic and non-genetic diseases of all kinds; nutritional diseases of all kinds; central nervous system diseases of all kinds, including psychiatric conditions; cancers and tumors of all kinds; cardiac diseases of all kinds; other tissue or organ diseases of all kinds; immunologic diseases of all kinds; addictive diseases of all kinds; other diseases of all kinds. Diagnostic tests for the above-described diseases. Products and services, which are associated with research and development associated with a disease or with the diagnosis, prevention, control, treatment, or cure, of a disease.

More specifically, the invention can be used in most steps in the overall process of human and veterinary drug and bioactive compound (BA) and biomarker (BM) development, which includes the development of antimicrobial, and antiviral agents as well as other drugs and BAs and BMs. Such steps include, but are not limited to, the following: the discovery and identification of drug bioactive compound(BA) or biomarker(BM) candidates; the evaluation of the specificity, toxicity, and efficacy, of drug BA or BM candidates; the development of drug or BA or BM candidate related diagnostic tests; the improvement and/or optimization of drug or BA or BM candidate's specificity, and/or toxicity, and/or efficacy, and/or pharmacokinetic characteristics; the identification of clinical screening participants and the drug or BA candidate market niche; quality control and quality assurance for drug or BA or BM production and manufacturing; the efficient prescription of drugs or BA for patients; the development of global and/or non-global particular protein expression profiles which are specific for prokaryotic and eukaryotic normal and abnormal cell states of all kinds including diseased and pathologic and chemically and/or physically and/or psychologically treated cell states of all kinds as well as differentiated and/or undifferentiated cell states of all kinds.

In addition the invention can advantageously be used in the characterization, quality control, and use, of organisms and their organs and tissues and cells, including primary cells and stem cells, as well as in vitro cultured organs and tissues and cells including, primary cultured cells and stem cells, for different aspects of the drug development process. This includes the use of gene knockout and other organisms, and their organs and tissues and cells, as well as in vitro cultured organs and tissues and cells, including primary cells and stem cells, and also includes interfering RNA treated gene knockout and their organisms, and their organs and tissues and cells, as well as in vitro cultured organs and tissues and cells, including primary cells and stem cells, for use in the different aspects of the drug development and use process.

The invention also is useful in industrial and applied applications which are related to biology. These include but are not limited to, the following. Many of the above-described applications for biological, agricultural, medical, and drug development areas of application which relate to water quality, food quality, public health, ecology, including environment and marine concerns, toxicology, forensics, diagnostics or many kinds, technology development, quality assurance and control. Also, standards for the development, production, or manufacture of applied products, and various services associated with the above areas of applications.

In accordance with the discussion above, the invention commonly involves improvement of differential protein expression (DPE) or differential gene expression ratio (DPER) results for a particular protein. Herein reference to particular protein DPER is typically shortened to PP-DPER. For prior art PP-DPER determination assays:
(i) It is well known that signification differences in the (total protein content/cell) and/or (the particular category of protein of interest content/cell) commonly occur for assay compared cell samples;
(ii) Prior art rarely if ever knows or determines the assay values for the (total protein content/cell) or (the particular category of protein of interest content/cell) for the assay compared cell samples, and does not normalize the prior art produced PP-DPER value for such protein content/cell differences);
(iii) The EA Rule is virtually always practiced;
(iv) Prior art practices and believes that the relationship (measured PP-DPER value)=(particular protein ACR value)=(particular protein T-DPER value).

When the EA Rule is practiced for a prior art PP-DPER assay determination, the above (iv) relationship is valid only if the number of sample cells or cell equivalents compared in the assay is equal to one. Thus, only when the ratio of ((the number of sample 1 cells or cell equivalents represented by the sample 1 protein present in the assay)÷(the number of sample 2 cells or cell equivalents represented by the sample 2 protein presents in the assay))=1, is the relationship (ACR=T-DPER) valid for the assay. Here, this assay sample cell number ratio is termed the assay SCR value. When, the assay SCR≠1, then the (ACR=T-DPER) relationship is not valid for a sample comparison particular protein DPER determination. When the (ACR≠T-DPER) then (the assay measured and normalized PP-DPER value)≠(particular protein T-DPER value). Further, when all other aspects of the protein expression comparison assay work perfectly, then the magnitude of the deviation of the assay SCR from one, is equal to the magnitude of the deviation of the particular protein ACR value from the particular gene T-DPER value, and the magnitude of the deviation of the measured PP-DPER value from biological correctness. Note that an extensive discussion of SCR≠1 situations and their effect on the biological correctness and interpretation of prior art measured particular gene differential gene expression ratio (DGER) values is presented in U.S. Provisional Patent Application number U.S. 60/687,526 (11) and the corresponding non-provisional application Ser. No. 11/421,961, both entitled “Method for Producing Improved Gene Expression Analysis and Gene Expression Analysis Comparison Assay Results”, both of which are incorporated herein by reference in their entireties. The described effects of assay SCR≠1 situations on the biological correctness and interpretation of prior art particular gene DGER values are essentially identical to the effect of assay SCR≠1 situations on the biological correctness and interpretation of prior art measured PP-DPER values.

Prior art protein expression comparison assay practice does not know or determine the assay SCR value, and does not normalize the assay measured PP-DPER values, for the SCR≠1 assay situations. Since naturally occurring significant differences in the total protein content/cell and particular category protein content/cell are common for compared cell samples, and the EA rules is almost universally practiced, it is very reasonable to believe that prior art protein expression assay SCR≠1 values quite commonly occur, and that many prior art protein expression assay measured PP-DPER values are significantly biologically incorrect. Further, it is reasonable to believe that the magnitude of deviation of the assay SCR from one, and the resulting magnitude of deviation of the assay measured PP-DPER vale from biological correctness, are significant relative to the measurement accuracy of the protein expression comparison assay.

Certain prior art protein expression assay measured PP-DPER values can be known to be biologically erroneous. For example rapidly growing E. coli cells have a total protein content/cell which is about 5 fold greater than the total protein content of slowly growing cells, and thus, for a comparison of rapidly and slowly growing E. coli cells, the assay SCR deviates from one by 4-5 fold (12). This is a very significant deviation from one, relative to the reported protein expression comparison assay measurement accuracy of ±1.2 to ±3 fold which has been reported (10). Since prior art does not know or determine the assay SCR values for protein expression comparison assays, it cannot be known whether other prior art protein expression comparison assay SCR values deviate from one or not, and therefore it cannot be known whether PP-DPER values associated with these assays are completely or incompletely normalized, or biologically correct or incorrect. As a result, these prior art produced PP-DPER values are uninterpretable with regard to the quantitative value and the direction of regulation implicit in the PP-DPER value. Because of the above situation, all prior art produced protein expression assay PP-DPER values can be known to be incorrect or uninterpretable.

Protein expression comparison assay SCR values must be determined in order to normalize for them. Reference 11 and the corresponding application Ser. No. 11/421,961 include extensive discussion concerning the factors involved in determining an assay SCR value for gene expression comparison assays and normalizing gene expression assay results for the number of sample cells analysed and the assay SCR value. These general considerations, rationales, and methods also apply to the determination of and the normalization for cell sample PP expression values and the assay SCR value for a protein expression comparison assay, and so will be only briefly indicated here. These considerations include but are not limited to, the following: (a) The total protein content/cell and/or particular protein category protein content/cell must be known for the intact cells of the compared cell samples. This can be determined using established methods for determining sample cell number and the amount of protein in a sample and the total protein content/cell, and certain particular categories of protein contents/cell. (b) For certain protein expression comparison assays the efficiency of isolation of the total protein and/or the particular protein category from intact cells must be known. Prior art methods exist for doing this and can be used for this purpose. (c) The assay SCR value must be determined from the amounts of each cell sample protein or protein fraction which is directly compared in the assay. For a 2-D gel assay this is the amount of each cell sample's protein loaded onto the gel. For mass spec analysis this is the amount of each cell sample protein actually analyzed in the mass spec assay. (d) The degree of degradation of each compared cell sample protein prep should be determined and taken into consideration when determining the assay SCR. This can be accomplished with established prior art methods.

To facilitate the normalization, it can be advantageous to use artificial housekeeping proteins. As described in the Background, prior art gene expression analysis studies often assume that for different compared cell samples certain particular genes (commonly term Housekeeping Genes (HG)) are present at the same RNA copy per cell numbers or abundance, i.e. a particular gene (PG) RNA in one cell sample has the same abundance as the same PG in the compared cell sample. This assumption is made to facilitate the normalization of gene expression assay results, but naturally occurring PG RNAs have not been validly demonstrated to occur.

Similarly, it has not been validly demonstrated that one or more certain particular proteins (PP) or HG PPs are naturally present at the same copy per cell or abundance in all or many compared cell samples analysed in protein expression analysis assays.

Thus, the determination and normalization of protein expression analysis results for the number of sample cells assayed and the SCR and other assay variables can be facilitated by the use of one or more control AHGPs in the assay. Reference 11 and the corresponding U.S. patent application Ser. No. 11/421,961 extensively discuss the creation and use of artificial housekeeping gene RNAs for the determination of and normalization for assayed sample cell numbers and SCR values for the production of improved gene expression analysis results. Those methods and rationales and uses discussed are also applicable for the creation and use of control AHGPs for the determination of and normalization for assayed cell sample number and SCR and other assay variables.

After determining the protein expression comparison assay SCR value, the assay determined PP-DPER values can be normalized or corrected for the effect of assay SCR≠1 situations by using the relationship (normalized assay measured PP-DPER value)=((assay measured PP-DPER value)÷(assay SCR value)). When, as prior art believes, the assay measured PP-DPER value is correctly normalized for all other assay pertinent variables, (the SCR normalized assay measured PP-DPER value)=(particular protein T-DPER value) and is therefore biologically correct.

Reference 11 and corresponding non-provisional application Ser. No. 11/421,961 extensively discuss the occurrence of gene expression comparison assay SCR≠1 related false negative gene expression results and their associated regulatory direction miscalls. Similar false negative protein expression results and their regulatory direction miscalls can occur for prior art protein expression comparison assays. The effect of the assay SCR≠1 value on the occurrence of such false negative results is essentially the same for both gene expression comparison results and protein expression comparison results, and is discussed extensively in reference 11 and application Ser. No. 11/421,961. Therefore, it will not be repeated here.

One General Example of the Practice of the Invention
(1) Obtain the cell samples to be compared.
(2) Prepare each cell sample protein preparation to be analysed.
(3) Determine the amount of each analysed cell sample protein preparation which represents one cell equivalent of protein.
(4) Analyse a known amount of each sample's protein preparation in the assay.
(5) Determine the number of each assay analysed sample's cells and/or the assay SCR value from the amounts of each analysed cell sample's protein preparation which is analyzed in the assay and the amount of each cell sample protein preparation which represents one sample cell.
(6) Determine the assay measured cell sample PP expression values of interest and/or the PP-DPER value for each particular PP of interest.
(7) Normalize each measured cell sample PP expression value and/or each PP-DPER value for the number of each sample's cells analysed in the assay and/or the assay SCR value to produce PP expression values and/or PP-DPER values which can be known to be improved relative to prior art practice measured and normalized PP-DPER values, by virtue of being normalized for the number of sample cells analysed and/or a known assay SCR value. Note that such SCR normalized PP-DPER values are improved even when the assay SCR=1 because the SCR normalized PP-DPER value is known to be correctly normalized for the assay SCR value.
(8) If desired, the improved PP expression results and/or PP-DPER results can be used for any other application which utilizes PP expression and/or PP-DPER results, to produce improved any other application results. These any other application improved results are improved, relative to such prior art any other application results, by virtue of being generated with improved PP expression and/or PP-DPER results.
(9) The improved application results can be used for further application which uses such any other application results to produce improved further application results.

A Second General Example of the Practice of the Invention Which Utilizes Control AHGP Molecules
(1) Obtain the cell samples to be compared.
(2) Prepare the protein sample to be analysed from each cell sample.
(3) Determine the amount of each analysed cell sample protein preparation which represents one cell equivalent for each analysed cell sample.
(4) Analyse a known amount of each sample's protein in the assay.
(5) Determine the number of each assay analysed sample's cells and/or the assay SCR value for compared cell samples from the amounts of each cell sample's protein preparation which is analysed in the assay and the amount of each cell sample protein preparation which represents one sample cell.
(6) Add known amounts or numbers of molecules of one or more different control AHGPs to each analysed cell sample protein preparation to create the presence in each analysed cell sample of one or more artificial housekeeping PPs(AHGPs) for which the abundance value is known for each analysed cell sample.
(7) Determine the assay measured PP expression value and/or PP-DPER value for each cell sample particular protein of interest and each control AHGP of interest.
(8) Normalize each measured and PP expression value or PP-DPER value for the number of sample cells analysed in the assay or the assay SCR value to produce cell sample PP expression values and/or PP-DPER values which can be known to be improved relative to prior art practice measured and normalized PP expression values and PP-DPER values, by virtue of being normalized for a known assay value for the number of sample cells analysed and/or assay SCR value. Normalization for the known number of analysed sample cells can be done utilizing the relationship: (the normalized assay measured cell sample PP expression value)=[(the assay measured cell sample PP expression value) divided by (the number of sample cells analysed in the assay)]. The SCR normalization can be done utilizing the following relationship: (the SCR normalized cell sample PP-DPER value)=(the assay measured cell sample PP-DPER value) divided by [(the assay measured expression ratio value for a control AHGP) divided by (the known control AHGP abundance ratio for the control AHGP for the cell sample comparison)]. Note that such PP-DPER normalized values are improved even when the assay SCR=1 because the SCR normalized PP-DPER value is known to be correctly normalized for the assay SCR value.
(9) If desired, the improved PP expression and/or PP-DPER results can be used for any other application which utilizes PP expression and/or PP-DPER results, to produce improved any other application results. These any other application improved results are improved, relative to such prior art any other application results, by virtue of being generated with improved PP expression and/or PP-DPER results.
(10) The improved application results can be used for further application which uses such any other application results to produce improved further application results.

Note that the practice of the invention can be used for same protein different cell (SPDS) and different protein different sample (DPDS) assay comparisons.

Note further that the above example is just one of many different practices of the invention. A large number of other examples exist which may differ in the normalization process or assay type as well as other factors.

Computer Implementation of Methods for Determining and Using Improved Assay Normalization Techniques

For the portions of the invention involving calculations, comparisons, analyses, and the like, the measurement, determination, and calculation of assay values, normalization of results for particular assays, use of the normalized results, and further analyses and determinations can be performed at least in part using software program or non-software program methods for calculating or determining the respective values. Advantageously, particularly for applications involving large amounts of data, such calculations are carried out using computers loaded with software for performing the various calculations and/or for displaying results. Persons skilled in the field are familiar with performing the relevant calculations, comparing and correlating and interpreting the resulting values, coding the functions in a suitable programming language, and configuring computers to implement the resulting programs and/or to display the relevant results in desired formats. Thus, the calculational steps will not be repeated here. A large number of programs have been developed for performing similar functions based on the types of assay and protein molecules. If desired, such software can be modified or extended to perform the present calculations.

Thus, the present invention also concerns such computer software, associated databases and data sets, and the use of computers running such software to implement at least portions of the present invention. Such software may be in hard copy (e.g., printing code and/or data) or may be embedded in one or more forms of computer accessible data storage such as random access memory (RAM), read only memory (ROM), magnetic storage media such as computer hard drives, tapes, and floppy disks, optical storage media such as CDs and DVDs and the like, and flash memory devices. The software may be in one or more portions (e.g., modules), which may be in the same physical storage device or in a plurality of different physical storage devices. Likewise, when loaded on a computer, the software may be accessible from a single computer, from any of multiple computers on a LAN or other local network or file transfer connection, or from any of multiple computers over the internet or a WAN or other large scale network. Therefore, the invention also concerns data storage devices and computer systems in which such software is loaded or stored, as well as methods using such software and computer systems to perform the designed functions of the software.

The various functions involved in the present determinations (as well as related determinations) can be performed by separate software programs or other methods, or can be embodied in a single software program or other method.

In many cases, utilization of the software will involves direct or indirect specification of assay conditions and requirements. The particular parameters which should or must be specified will depend on the particular application and assay times.

Databases & Data Sets

Advantageously, one or more databases (or data sets) can be used which contain data on items used in the respective calculations. Several different types of data which can be advantageously included in such databases are pointed out below. However, a database or set of linked databases need not include all the indicated data in order to be useful, and may include additional data not mentioned. Further, in some implementations, experimental data may be used to derive or otherwise obtain an algorithm at least approximately describing one or more effects (e.g., effects listed below), such that use of the algorithm (e.g., manually or as part of a computer program) may replace use of a corresponding database for at least some range of assay variables. Likewise, when linked with a computer program, a program may be configured to interpolate between data points (e.g., using any of a variety of known and available interpolation algorithms) to approximate effects for conditions which are not exactly or not completely represented in the database.

Sequence and Sequence-Related Data: One such database (or set of databases) or data set (of set of data sets) contains sequence and/or sequence related data for the protein of interest, e.g., for a particular cell type of interest. Such a database can, for example, include sequence information for proteins from a set of a plurality of genes, or from all or essentially all expressible genes in cell. (For purposes of this discussion, unless clearly indicated to the contrary, reference to a database shall include one or more databases (e.g., one or more databases accessible from a computer or computer system), and shall also include the data sets stored in the database. Further, at least some of the information may be in publicly accessible databases, such as in GenBank and related or similar databases.

Likewise, such database may contain such sequence information for a plurality of different types of cells. For example, such cells may be from various source organisms (e.g., human, mouse, rat, pig, ape, monkey, or other non-human mammal, bacteria, yeast), may be from different tissues in an organism or organisms), may be from a cell line (e.g., an immortal or immortalized cell line) may be normal, may contain gene variants (e.g., allelic variants, splice variants, mutations, and the like), may be pathological or diseased (e.g., cancer or other neoplastic cell), may be infected with one or more microorganisms (e.g., viral, bacterial, or other microorganism), may have been treated with one or more chemicals and/or particular physical conditions, may be from an organism which has been treated with or subjected to one or more particular chemical, drug, and/or environmental conditions, and/or may be prokaryotic or eukaryotic, among others.

Such database can contain information on the partial or complete gene expression profiles for one or more or all cell sample RNAs and/or proteins which are associated with one or more different cell samples or cell sample treated or physiological states.

Such database can contain information on variants and processed forms of particular genes, RNA produced from those genes, e.g., allelic variants, mutants with detectable phenotypic effect, splice and other RNA processing variants, homologous forms, and the like, and/or expressed protein sequences for such variants.

Such database can include data describing the amino acid sequence, length, and/or amino acid composition of particular proteins, peptides, and/or capture molecules for specifically binding to particular proteins. Thus, for example, the database can include such data for the proteins or peptides in a microarray (preferably for each spot of interest).

Binding and reaction properties: Likewise, a database may contain data describing the binding properties and/or reaction properties for particular proteins and/or between particular proteins and other molecules. Such data may be for unlabeled and/or labeled (directly or indirectly) protein and/or specific protein binding molecules.

Protein Degradation Parameters: Data related to protein degradation (e.g., due to protease cleavage during sample preparation) can also be useful. For example, in relation to undegraded sample protein and degraded sample protein, data describing or characterizing the extent and location of cleavage for particular proteins in particular cell sample types can be beneficial.

Program Functions

The software program can be readily configured as desired to provide appropriate functions for the intended application.

In particular application it will be desired to calculate assay values for assay SCR and to apply assay SCRs to normalize assay results. In addition, in many case it will be desired to carry out additional analyses or determinations, with the particular analyses or determinations depending on the particular application. Examples of types of analysis techniques and methods which can be implemented and used include those described for various aspects of the invention.

Improved Protein Array Kits for Performing Assays

Practice of the methods described above for improved normalizing of a variety of different assays can involve changes or additions in the materials used for performing the assays or in performing associated determinations relating to improved normalization, validation, calibration, and/or corroboration of assay results. Components and/or instructions for carrying out processes can be useful incorporated and supplied in kit form, e.g., an assay kit with additional components and/or instructions for performing the further functions. Alternatively, separate assay kits can be provided for performing the improved normalization, validations, or corroboration of separate assay results. In most cases, the kits will be packaged or otherwise assembled together. A kit may be single use, but in many case will have sufficient components for carrying out multiple assays, e.g., at least 2, 3, 5, 10, 20, 50, or 100 such assays.

Thus, in many cases, the kit will include one or more components for carrying out the assay, along with instructions and/or materials for carrying out improved normalization and/or for determining that a normalization process is improved or valid and/or for calibrating the assay and/or for corroborating results for basic assay and/or for evaluating the performance characteristics of an assay. Such instructions may be in various forms, e.g., written and/or graphic and/or electronic, and one or more forms may be used for a particular assay kit. Electronic forms may be provided directly, or may be provided in the form of directions for accessing the instructions (e.g., internet site access directions). Either as part of the instructions or separately, computer software for carrying out improved normalization and/or the other functions indicated herein can be supplied.

Similarly, in many cases the kit will alternatively or additionally include one or more AHGPs for use in the assay and/or instructions for using such AHGPs in the assay (which may be separate from other instructions for carrying out the assay or may be combined with other such instructions. Such AHGPs may, for example, be supplied separately in single use form or in multi-use or bulk form, or may be supplied as a mixture of different AHGPs in single use form or multi-use or bulk form. Electronic forms of instructions may be used as indicated above.

The invention also concerns the instructions separately. For example, such instructions may be provided on a web site or in the form of a printed or electronic manual, e.g., a book or booklet, which may contain instructions for additional assays, information on assay systems, evaluation reports, and/or other information, or as included information in a catalog or similar format.

Those familiar with such assays are familiar with components which are commonly included in commercial assay kits, such as microarrays, for example, binding solutions, wash solutions, detection solutions, labeled detection molecules, and the like.

As indicated, the kit can include physical components used in the assay and/or components for determining improved normalization factors related to the assay. Those components will depend, in part, on the type of assay for which the kit is intended, e.g., a microarray assay, a 2D gel electrophoresis assay, an affinity binding media method, a mass spectroscopy method, an immunoassay method, an ELISA method, and the like.

Thus, a large number of useful assay kits can be constructed which provide the present assay improvements and/or corroboration. All such assay kits are within the present invention.

Exemplary Applications and Techniques for Improved Protein Expression Assay Results

A substantial number of different patent documents concerning applications involving protein expression assays and/or applications of the results of such assays (directly or indirectly) are listed below. These patent documents are illustrative of the state of the art, provide exemplary description for carrying out techniques involved in the practice of the invention, and provide exemplary applications and thus exemplary embodiments for application of the present invention. Thus, each of the U.S. patent documents in the following list is incorporated herein by reference in its entirety.

U.S. patent application Publ. 2001/0039016

U.S. patent application Publ. 2002/0012905

U.S. patent application Publ. 2003/0032017

U.S. patent application Publ. 2003/0054367

U.S. patent application Publ. 2003/0064397

U.S. patent application Publ. 2003/0124548

U.S. patent application Publ. 2003/0148295

U.S. patent application Publ. 2004/0023306

U.S. patent application Publ. 2005/0048566

U.S. patent application Publ. 2005/0095592

U.S. patent application Publ. 2005/0074937

U.S. patent application Publ. 2005/0112706

U.S. patent application Publ. 2005/0221398

U.S. patent application Publ. 2005/0233399

U.S. patent application Publ. 2006/0029574

U.S. patent application Publ. 2006/0035239

U.S. patent application Publ. 2006/0068452

U.S. Pat. No. 6,670,194

U.S. Pat. No. 6,852,544

U.S. Pat. No. 6,897,020

U.S. Pat. No. 6,921,642

U.S. Pat. No. 7,069,151

### EXAMPLES

The following examples are drawn from selected documents from the list above, and illustrate applications in which the present improved results can be utilized, and techniques for carrying out the illustrated applications as well as others. Certain of the applications and describe the use of results from nucleic acid based gene expression assays and comparisons. Advantageously, such results and applications utilizing those results are improved in accordance with U.S. application Ser. Nos. 11/421,961 and/or 11/453,298, both of which are incorporated herein by reference in their entireties.

### Example 1

### Example 1 from U.S. patent application Publ. 20030124548

This example illustrates the use of nucleic acid-based gene expression assays, which will benefit from application of improvement from proper normalization, applied in the context of subsequent protein expression comparisons. The particular work described demonstrates gene induction by ligand-stimulated receptor tyrosine kinases (RTKS) in fibroblast cells.

Receptor Tyrosine Kinases (RTKs) transduce extra-cellular signals that trigger important cellular events, such as mitosis, development, wound repair, and oncogenesis. When bound by ligand(s), RTKs mediate these responses by activating a variety of intracellular signaling pathways. Such signaling pathways result in the transcription of a set of “Immediate Early Genes” (IEGs). IEG products initiate cellular processes that depend on protein synthesis, such as mitogenesis. Wild-type and mutant strains of NIH3T3 mouse fibroblast cells are stimulated with macrophage-colony stimulating factor (M-CSF) for various time points, and the M-CSF-activated signaling pathway-induced gene expression is determined. The essential objective of the study is to characterize the RTK-mediated interactions between the intracellular signaling pathways.

Experimental Methodology

The following equipment used for experiments in this Example includes an Ohaus Explorer analytical balance, (Ohaus Model #EO1140, Switzerland), biosafety cabinet (Forma Model #F1214, Marietta, Ohio), pipettor, 100 to 1000 μL (VWR Catalog #4000-208, Rochester, N.Y.), cell hand tally counter (VWR Catalog #23609-102, Rochester, N.Y.), CO₂ Incubator (Forma Model #F3210, Marietta, Ohio), hemacytometer (Hausser Model #1492, Horsham, Pa.), inverted microscope (Leica Model #DM IL, Wetzlar, Germany), pipet aid (VWR Catalog #53498-103, Rochester, N.Y.), pipettor, 0.5 to 10 μL (VWR Catalog #4000-200, Rochester, N.Y.), pipettor, 100 to 1000 μL (VWR Catalog #4000-208, Rochester, N.Y.), pipettor, 2 to 20 μL (VWR Catalog #4000-202, Rochester, N.Y.), pipettor, 20 to 200 μL (VWR Catalog #4000-204, Rochester, N.Y.), PURELAB Plus Water Polishing System (U.S. Filter, Lowell, Mass.), Refrigerator, 4° C. (Forma Model #F3775, Marietta, Ohio), vortex mixer (VWR Catalog #33994-306, Rochester, N.Y.), a water bath (Shel Lab Model #1203, Cornelius, Oreg.), microfuge tubes, 1.7 mL (VWR Catalog #20172-698, Rochester, N.Y.), pipet tips for 0.5 to 10 μL pipettor (VWR Catalog #53509-138, Rochester, N.Y.), pipet tips for 100-1000 μL pipettor (VWR Catalog #53512-294, Rochester, N.Y.), pipet tips for 2-20 μL and 20-200 μL pipettors (VWR Catalog #53512-260, Rochester, N.Y., pipets, 10 mL (Becton Dickinson Catalog #7551, Marietta, Ohio), pipets, 2 mL (Becton Dickinson Catalog #7507, Marietta, Ohio, pipets, 5 mL (Becton Dickinson Catalog #7543, Marietta, Ohio) and a cell scraper (Corning Catalog #3008, Corning, N.Y.)

Chemicals, reagents and buffers necessary include dimethylsulfoxide (DMSO) (VWR Catalog #5507, Rochester, N.Y.), Modification of Eagle's Medium (DMEM) (Mediatech Catalog #10-013-CV, Herndon, Va.), fetal bovine serum, Heat Inactivated (FBS-HI) (Mediatech Catalog #35-011-CV, Herndon, Va.), Penicillin/Streptomycin (Mediatech Catalog #30-001-CI, Herndon, Va.), murine fibroblast cells (American Type Culture Collection Catalog #TIB-71, Manassas, Va.), tissue culture plate, 24-well, 3.4 mL capacity (Becton Dickinson Catalog #3226, Franklin Lanes, N.J.) and ultra-pure water (Resistance=18 megaOhm xcm deionized water).

Murine 3T3 cells (ATCC Number CCL-92) are grown in DMEM with 10% FBS-HI with added penicillin/streptomycin and maintained in log phase prior to experimental setup. To make growth medium, to a 500 mL bottle of DMEM, add 50 mL of heat inactivated FBS and 5 mL of penicillin/streptomycin. Store at 4° C. Warm to 37° C. in water bath before use.

Cell Surface Receptor Modification

A chimeric growth factor receptor having the signaling activity of M-CSFR and activated by binding macrophage colony stimulation factor (M-CSF), referred to as “wild-type” chimeric receptor (ChiR(WT)) is constructed using standard procedures in molecular biology. Also, a mutant strain ChiR(F5)-3T3 is constructed employing accepted site-directed mutagenesis techniques.

Gene induction in the wild-type strain is determined. ChiR(WT)-3T3 cells are stimulated with M-CSF alone and in combination with cycloheximide (CHX) to assess which induced genes behave as IEGs and which require protein synthesis for their induction. M-CSF treatments were 40 ng/ml in 0.5% bovine calf serum media for 20 min, 1 hr, 2 hr and 4 hr. CHX treatments were 10 μg/ml for 4 hr. Gene induction in the mutant strain is also determined. The F5 mutant strain is stimulated with M-CSF for 20 min, 1 hr, 2 hr and 4 hr.

Gene expression levels are measured using oligonucleotide arrays (Affymetrix) containing detectors for 5938 mouse genes and EST sequences. To be classified as an IEG in the wild-type strain, genes had to be induced by M-CSF in the presence and absence of CHX. Sixty-six genes met the criteria for being IEGs and an additional 43 genes are induced by M-CSF+CHX but are not strongly induced by M-CSF alone.

The RNA is used for expression monitoring, using oligonucleotide arrays (Affymetrix, Inc.) containing detectors for 5938 genes and EST sequences (FIG. 2). It should be noted that although changes in transcript abundance are not necessarily due to transcriptional upregulation, previous experiments have shown that transcriptional upregulation is by far the preponderant model if IEG induction by RTKs.

To initially identify a set of clear IEGs, stringent criteria are set including at least a 2-fold induction in both replicate studies and at least 3-fold induction in one of the replicates at one time point. Although the oligonucleotide arrays monitor less than 10% of the total number of mouse genes, the 66 IEGs probably represent a much larger proportion of the total number, because of extensive discovery efforts for this class of genes.

Protein quantification was determined from cell lysates using a Packard FluoroCount Model #BF10000 fluorometer (Meriden, Conn.). Other equipment not previously listed included a Forma Model #F3797-30° C. freezer, Heating Block (VWR Catalog #13259-030, Rochester, N.Y.), Microfuge (Forma Model #F3590, Marietta, Ohio). The procedure described in the NanoOrange Protein Quantitation Kit (Molecular Probes Catalog #N-6666, Eugene, Oreg.) is followed without modification.

Gene expression profiles were analyzed using iterative global partitioning clustering algorithms and bayesian evidence classification to identify and characterize clusters of genes having similar expression profiles. Since the dynamics of the expression profiles are important in elucidating the functional role of the genes, the entire time series of expression measurements for each gene was used in the analysis.

The steps involved are as follows:
1) Determine the fold induction (log ratio) of the genes in the wild-type and mutant strains for each time point relative to the control at 0 hr (no stimulation)
2) Normalize gene profiles to magnitude equal to 1
3) Conduct partitioning clustering on 6312 genes in each strain to determine unique clustering patterns
4) Differentiate gene clusters in each strain into the following sub-groups based on their expression as compared to the population-average profile: early up-regulated, late up-regulated, down-regulated and others
5) Carry-out a comparative analysis to explore the common genes in the early up-regulated and down-regulated cluster sub-groups in the two strains
6) Conduct correlation analysis based on the Pearson correlation coefficient to determine differences and similarities among the IEGs in the two strains.

Intermediate Early Genes Induced by M-CSF Treatment of NIH3T3 Cells

The IEGs induced by 40 ng/mL M-CSF stimulation of quiescent NIH3T3 WT and F5 mutant cells are listed in Table 2.1 of the cited patent publication by time of peak observed induction. Each gene is classified as previously reported if it has been reported to be M-CSF or serum inducible in fibroblasts.

Clustering of Gene Expression Profiles

Agglomerative algorithms such as hierarchical clustering start with each object (gene) being in a separate class. At each step, the algorithm finds the pair of the “most similar” objects, which are then merged in one new class and the process is repeated until all objects are grouped. Agglomerative algorithms produce a very large number of clusters when several thousands objects are involved in the data set.

One common problem with the interpretation of clustered data is to determine the “true” number of clusters. Agglomerative algorithms do not offer explicit “stopping rules” for determining the globally optimal number of classes but rather present the entire set of clusters to the user, who then has to decide on the proper degree of structure in the data.

In this example, we have used a partitioning k-means clustering algorithm to cluster the gene expression profiles iteratively into a maximum of 50 classes. This algorithm can produce a globally optimal solution since it starts with the entire data set. At each step of the algorithm the least homogeneous cluster is sub-partitioned and the process is repeated until a criterion for cluster “compactness” is met. Cluster homogeneity, or compactness, is based on the concept of fitness. The later is defined as the sum of distances observations from their corresponding cluster centroid, or 1 Fitness (C)=k=1 C i=1N k d(X ik, X k_)(1) where Xᵢₖ is the I-th observation vector assigned to the k-th cluster, Xₖ is the vector of the k-th cluster centroid, Nₖ is the number of observations, or size, of the k-th cluster, C is the number of clusters, and d(x,y) is the distance metric (typically the Euclidian distance) between two vectors. The fitness is largest for C=1 (entire population) and monotonically approaches zero as C approaches N, the total number of observations.

Cluster homogeneity is defined now as: 2 H (c)=[1−Fitness(c)Fitness(1)]×100(2) that takes asymptotically the value of 100%. The optimal number of clusters C*<N is found at a homogeneity level of less than 100, depending on the internal structure of the data.

The cluster homogeneity results from clustering of the gene expression data for the wild-type and mutant strains are shown in FIG. 3 of the cited patent publication. For the given settings, the algorithm arrives at an optimal number of 35 clusters.

Wild-Type Strain

Genes are grouped in 35 clusters, which ranged in size between 2 and 2719 genes per cluster. A measure of the average expression level of the genes in each cluster, as expressed by the Euclidean length of the cluster centroid, is shown as a function of cluster size in FIG. 4 of the cited patent publication.

As can be seen from the plot, a very large cluster consisting of 2179 genes (43.1% of total) exhibited expression levels almost identical to the control (length=0). On the other end, only 4 small clusters each containing at most 4 genes exhibited high expression levels throughout the time course (length>2). Finally, most of the gene clusters have moderate expression levels (length<1) and fall in the middle of the chart with size ranging between 50 and 200 genes per cluster.

Clusters are further sub-divided into the following categories based on their expression patterns: (1) early up-regulated (higher induction than population mean at 20 minutes); (2) late up regulated (higher induction than population mean from 1 hour onwards); (3) down-regulated (lower induction than population mean); and (4) others. The typical expression “signatures” for clusters in the above three categories are shown in FIG. 5 of the cited publication.

Early up-regulated genes exhibit a high level of expression at 20 min, indicating that these genes are IEGs, i.e. their induction does not require protein synthesis but involves latent transcriptional activators already present in the cell. Transcription of genes falling in the second category of late up-regulated genes most likely requires protein synthesis, as the expression level of these genes peaks after 1 hr from the stimulation event. Equally important are the genes falling in the last category whose expression was repressed as a result of stimulation by the extracellular signal.

FIG. 6 of the cited publication shows the relative size of the clusters of genes falling in the above categories. Only 13 genes (0.2%) are early up regulated, whereas a significant number of 481 genes (7.6%) are down regulated as a result of the treatment.

F5 Mutant Strain

Comparison of the expression profiles of the wild-type strain with the mutant strain F5, which carries tyrosine to phenylalanine mutations at key binding sites for critical signaling molecules, provides some important insight regarding the degree of overlap and interaction of the various regulatory pathways.

The expression data from the mutant strain are analyzed in the same way. The expression patterns are similar to those of the wild type strain resulting in 34 clusters. The cluster sub-groupings for the two strains are compared in Table 1.2 of the cited publication.

Interestingly, a similar number of genes are induced for both strains in response to the stimulant, but a larger number of genes in repressed in the mutant strain. Furthermore, it appears that the expression pattern of a larger number of genes is affected in the mutant strain compared to the wild type. This could indicate the activation of alternate or reserve pathways to compensate for the disruptions caused by the mutations.

Table 1.3 of the cited publication summarizes the expression profiles and functional annotations of the identified early up-regulated genes for each strain. As expected, most genes in this group code proteins that are either transcription factors or cytoplasmic regulatory proteins.

Comparison of the early-induced genes between the two strains is shown pictorially in FIG. 7(a) of the cited publication. Nine of the 13 IEGs (69%) were common between the two strains. In all, we observed differential expression patterns in 6 IEGs: 4 IEGs from the WT strain were not induced in F5, whereas a new set of two IEGs was observed in the mutant strain. This indicates that alternate signaling pathways might be active to transduce signals and activate the early response genes. However, these pathways seem to highly overlap.

Although the early transcriptional response of the two strains is very similar, the late up-regulated genes show a considerably lesser degree of overlap (see FIG. 7(b) of the cited publication). The total number of genes following a late up-regulated induction profile is remarkably similar between the two strains, but only 44 (18%) were common genes, showing a great diversity in response pathways. Also, there were 215 (26%) common genes among the down-regulated clusters.

Finally, correlation analysis of the early up-regulated genes for the two strains is carried out to evaluate similarities in the expression profiles of the entire 15 genes. As shown in FIG. 6 of the cited publication, there is a strong correlation between the same genes in the two strains (diagonal of the array), even for those genes that are classified as belonging to the IEGs for only one of the two strains (compare with FIG. 7(a) of the cited publication). Furthermore, the non-common IEGs can be discerned based on differences in their expression patterns relative to the other genes. These are concentrated towards the lower correlation quadrants of the array (top right corner).

The tools of clustering and correlation analysis are shown to be valuable in identifying and characterizing subtle differences in the expression profiles of biological systems. These techniques would potentially impact comparative genomics studies, especially when proteomic data are available for further elucidation of physiological pathways.

Signaling Pathways Within Clusters of Early Up-Regulated Genes

Using the prior art, it is demonstrated that current programs for signaling network analysis lack the functional dimension of the present invention. This deficiency limits the success of any pathway-finding program when using newly developed data rather than data from known pathways. The pathway finding operation described at http//geo.nihs.go.jp/csndb/batch_search.html, is used within the gene clusters for early up-regulated genes listed in Table 1.4 of the cited application. Although the database contains only human pathways, the proteins identified by the gene cluster analysis are all listed in the database, indicating human analogs.

The batch search for pathways found no pathways for cluster 12, 19, 20 or 35 gene expression data. This negative result is expected for the reasons previously discussed. The lack of functional data significantly limits the breath of inference from gene expression data. As indicated in Example 2 of the cited publication (below), however, the addition of even small data sets of functional data dramatically increases the information derived from gene microarray experiments.

### Example 2

### Example 2 from U.S. patent application Publ. 20030124548

This example delineates the physiological processes and signaling pathways activated through growth factor receptors. This example illustrates that gene expression and proteomic data gathered following cellular stimulation can be interpreted in mechanistic terms by comparing the gene expression profiles to post-translational modifications of proteins with algorithms for determining linkages and associations. Such linkages and associations are then useful for identifying critical cellular pathways employed in complex cellular response mechanisms. As indicated above, the protein expression and proteomic data can be improved by application of the present normalization improvements.

Methods

General methods for cell culture, stimulation, and preparation of RNA are performed as described in Example 1 of the cited publication. Additional equipment for proteomic analysis is described.

Equipment for SDS-PAGE includes a Mini Vertical Gel System (Savant Model #MV120, Holbrook, N.Y.) and power supply (Savant Instruments Model #PS250, Holbrook, N.Y.). Supplies and reagents for western blotting are 10-20% precast gradient mini-gels (BioWhittaker Molecular Applications Catalog #58506, Rockland, Me.), 2× sample buffer (Sigma Catalog #L-2284, St. Louis, Mo.), beaker, 1000 mL (VWR Catalog #13910-289, Rochester, N.Y.), color molecular weight standard (Sigma Catalog #C-3437, St. Louis, Mo.), glycine (Sigma Catalog #G-7403, St. Louis, Mo.), graduated cylinder, 1000 mL (VWR Catalog #24711-364, Rochester, N.Y.), microfuge tubes, 0.5 mL Safe-Lock (Brinkmann Catalog #22 36 365-4, Westbury, N.Y.), microfuge tubes, 1.7 mL (VWR Catalog #20172-698, Rochester, N.Y.), pipet tips for 2-20 μL and 20-200 μL pipettors (VWR Catalog #53512-260, Rochester, N.Y.), pipet tips, gel loading (VWR Catalog #53509-018, Rochester, N.Y.), sodium dodecyl sulfate (SDS) (Sigma Catalog #L-4509, St. Louis, Mo.), Stir Bar, Magnetic (VWR Catalog #58948-193, Rochester, N.Y.), storage bottle, 1000 mL (Corning Catalog #1395-1L, Corning, N.Y.), and trizina Base (Sigma Catalog #T-6066, St. Louis, Mo.).

Prepare 5×SDS-PAGE buffer by dissolving 15 grams of Tris base, 72 grams glycine, and 5 grams SDS in 900 mL distilled water in a 1000 mL beaker with a magnetic stir bar. Place on a magnetic stirrer and stir until dissolved. Adjust volume to 1000 mL with a 1000 mL cylinder. Store at 4° C. Prepare 1×SDS-PAGE buffer by combining 200 mL of the 5× stock with 800 mL water. Store in a 1000 mL storage bottle at 4° C. Warm to room temperature before use. Melt the 2× Sample Buffer at room temperature and store as 500 μL aliquots in 1.7 mL microfuge tubes in −30° C. freezer. Assemble vertical gel system according to manufacturer's guidelines. Pour enough 1×SDS-PAGE buffer into gel system to cover top of gel and enough in bottom of the apparatus to cover bottom of glass plates. Remove a tube of 2× Sample Buffer from freezer and melt at room temperature. Melt frozen cell lysate samples on ice. Dilute cell lysate samples 1:1 with 2× sample buffer in 0.5 mL Safe-Lock tubes (15 μL of cell lysate sample and 15 μL 2× Buffer). Put remaining 2× Sample Buffer back into freezer (−30° C.). Put cell lysate samples back into freezer (−80° C.). Heat protein samples and molecular weight standards (if required) to 95-100° C. for 5 minutes. Briefly, spin in microfuge to collect sample at bottom of tube, and load equal amounts of protein in wells of pre-cast gel. Run at 30 mA per gel at constant current for 60 minutes or until dye reaches the bottom of gel.

Supplies and reagents for western blotting of phosphotyrosyl proteins includes anti-phosphotyrosine antibody 4G10 (UBI Catalog #05-321, Lake Placid, N.Y.), Blotting Paper (VWR Catalog #28303-104, Rochester, N.Y.), glycine (Sigma Catalog #G-7403, St. Louis, Mo.), hydrochloric acid (HCl) (VWR Catalog #VW3110-3, Rochester, N.Y.), methanol (VWR Catalog #VW4300-3, Rochester, N.Y.), NaOH (Sigma Catalog #S-5881, St. Louis, Mo.), nitrocellulose membrane (Schleicher & Schuell Catalog #10402680, Keene, N.H.), Nonfat dry milk (Carnation Brand), peroxidase labeled goat anti-mouse IgG (KPL Catalog #474-1806, Gaithersburg, Md.), and phosphate buffered saline (PBS) (Mediatech Catalog #21-040-CV, Herndon, Va.).

Perform SDS-polyacrylamide gel electrophoresis for phosphotyrosine proteins on cell lysate sample as in Example 1. Remove membrane from glass plates and equilibrate in Towbin buffer for 5 minutes with gentle rotation at room temperature. Cut nitrocellulose membrane to correct size, nicking off the lower right hand corner. Prewet membrane with ultra-pure water, then equilibrate for 5 minutes in transfer buffer. Prewet 6 pieces of blotting paper for each gel to be transferred in 1× Towbin buffer.

Set up transfer sandwich according to the manufacturer's directions. Transfer proteins at 96 mA per gel for 60 minutes per gel. Check for good protein transfer by staining with 10 mL Ponceau S solution for 5 minutes, then washing several times with water. Block the blotted membrane with 10 mL of freshly prepared PBS containing 3% nonfat dry milk (PBS-NFDM) for 20 minutes at room temperature with constant agitation. Incubate the membrane with the primary antibody diluted to 1 μg/mL in 5 mL freshly prepared PBS-NFDM overnight at 4 degrees C. and sealed in a plastic bag.

Wash the membrane twice with water. Incubate the membrane in the secondary antibody diluted 1:3000 in 10 mL freshly prepared PBS-NFDM for 1.5 hours at room temperature with constant agitation. Wash the membrane twice with water. Wash the membrane in PBS-0.05% Tween 20 for 3.5 minutes at room temperature with constant agitation. Wash membrane 3-4 times with water. Detect tyrosine phosphoproteins using chemiluminescence.

Chemiluminescence for visualization of phosphotyrosine proteins is performed using a UVP darkroom with cooled integrated camera (Epi Chemi II Darkroom with LabWorks Software, UVP, Upland, Calif.), LumiGlo® Chemiluminescent Substrates A and B (KPL Catalog #54-61-02, Gaithersburg, Md.). Remove LumiGlo® Chemiluminescent Substrates A and B from refrigerator. After proteins have been blotted to nitrocellulose or PVDV, drain excess water from membrane by touching edge of membrane on a clean KimWipe. Place membrane into a clean weigh boat or other suitable container. Add 0.8 mL of Substrate A and of Substrate B directly to membrane and swirl around to mix. Put LumiGlo® Chemiluminescent Substrates A and B back into refrigerator. Allow substrate to incubate on membrane for 1 minute at room temperature. Remove membrane from weigh boat, drain off excess substrates, and place directly onto the transilluminator of the Epi Chemi II system. In the LabWorks program provided, select On-Chip Integration and integrate for various times until a good signal is achieved (1, 3, 6, 10 and/or 15 minutes, depending on how much protein of interest is present on membrane). Using the software, identify bands of interest and print out the Integrated Optical Density of these bands.

Data Analysis:
1. For each protein band intensity measurements are first normalized to magnitude of 1 across the time profile. Data can also be normalized across protein bands to a magnitude of 1 at each time point.
2. Partitioning k-means clustering is applied to the normalized data as explained in Example 1 of the cited publication (above). Optimal number of clusters was determined to be 5.
3. Average profiles are calculated for the proteins within each cluster.
4. Protein clusters are grouped according to the dynamics accumulation to early or late phosphorylated clusters.

The similarity of the proteomic clusters to the genomic expression clusters is then determined through association analysis based on a similarity measure, as for example the Pearson's correlation coefficient or Euclidean distance of the two profiles.

Clustering of Proteomic Profiles

The k-means algorithm determined an optimal number of 5 clusters. The distribution of the proteomic clusters is shown in FIG. 2.1 of the cited publication.

Cluster A is the largest cluster containing 11 of the 21 visible phosphorylated protein bands. Cluster B is the smallest containing only 1 protein band, which has a unique profile compared to the other bands (see FIG. 2.2 of the cited publication).

The results of the clustering algorithm indicated that the phosphorylation profiles of all proteins were the most dissimilar at 1 and 2 h, and the most similar at 4 h. This clearly has implications on experimental design in this system, suggesting that if a single time-point design is to be followed, the proteomic measurement should be taken at 1 or 2 h after stimulation.

The time profiles of the phosphorylated protein clusters are shown in FIG. 2.2 of the cited publication. The total amount of phosphorylated protein (sum of intensity of all bands) is also shown for comparison. As can be seen, clusters E and C contain proteins that are phosphorylated as early as 20 min after addition of the stimulant. In particular, cluster E contains three proteins with molecular weights 93.3, 76.4 and 50.8 kDa that seem to have a role in the early stages of the signal transduction process.

Association Analysis of Gene and Proteomic Profiles

Separate analyses of gene expression and proteomic data resulted in classification of the different genes and phosphorylated proteins according to the dynamic profiles of their levels after stimulation with M-CSF. The gene expression clusters in particular identified groups of genes that showed high levels of induction, prior to protein synthesis. Similarly, two of the protein clusters showed early phosphorylation, suggesting that these proteins might be related somehow to the immediate early induced genes. If this analysis is extended to the entire set of gene expression and proteomic clusters, the association between protein phosphorylation and gene expression can be mapped out.

In the following analysis, the similarity of the gene expression and proteomic profiles was evaluated based on Pearson's correlation coefficient, which is defined as: 3 XY=1 N i=1 N(X i−X_s X)(Y i−Y_s Y)(3) where X is the expression profile of a gene cluster, Y is the expression profile of a protein cluster, N is the number of time points, and X̅ and sₓ are the average and standard deviation of the values in each profile.

The results of this analysis are shown in FIG. 2.3 of the cited publication. The figure shows the color-coded map of associations. The actual values of the correlation coefficient are also shown. To make the visual inspection clearer, the resulted correlation matrix was clustered in both directions and the rows and columns were re-arranged according to the results of the clustering.

From the visual inspection of the proteomic-genomic association matrix several areas of positive (red) or negative (green) association between the clusters is evident. For example, gene clusters 12, 20, and 35, which are early-regulated clusters, show a negative association with protein cluster A, indicating opposite regulation. Also, gene cluster 9 (containing 56 genes) shows a strong positive association with protein clusters C and E.

Further analysis of cluster 9 gene products with cluster E proteins using our protein database indicates an association of M-CSF with early response proteins PTP-1C and Shc. Both of these proteins are cytoplasmic tyrosine phosphatases. In our protein dataset, a network signaling linkage from PTP-1C is identified with the tyrosine phosphorylation of a 65-kDa cytoplasmic protein pp65.

Estimating signaling associations among signaling pathways within the gene cluster 9 and protein cluster E overlap, it is discovered that the highest degree of association (0.125) is achieved with cell cycle regulatory proteins (see FIG. 14 of the cited publication). These include cyclins D1, D2, D3 and E, cyclin dependent kinases CDK4/6/2 and RB protein. While further analyses of time sequences is not presented, an interesting strong down-regulation of the p53 protein is identified by the present invention at 1 hour followed by a stronger up-regulation by 4 hours.

As a knowledge-based system, information of associations in one series of experiments may be combined with other experiments to continue to improve the strength of association of adjacent molecules and pathways. Other post-translational processes added to the experimental design will also function to improve the strength of pathway identifications. This example illustrates that the combination of gene expression data and structure/function protein assessment with a structure/function protein database described by the present invention generates superior information relating to signaling networks and is more useful to the discovery of novel pathways.

### Example 3

### Example 1 from U.S. patent application Publ. 20020012905

Selecting Chemical Compounds for Toxicity Screening

Compositions that fall into particular categories of toxicity are used to establish molecular profiles and compile libraries for particular toxicities. Table 1 of the cited publication lists a number of compositions that are known to be toxic to certain tissues or organs or during developmental stages. In particular, those compositions that cause liver toxicities are assessed for their molecular profiles by determining alterations of gene or protein expression patterns in LSCs contacted by each composition. A library comprising molecular profiles of compositions having liver toxicities is therefore compiled. Those compositions causing cardiovascular toxicities are similarly assessed for their molecular profiles and a library compiled. In addition, molecular profiles and library thereof for compositions having toxicities on the central nervous system and for compositions having developmental toxicities are similarly established using the LSC system. The experimental procedures as described above in general, and in more detail in the following examples, are followed to compile the molecular profiles and libraries for compositions with particular type of toxicities.

Drugs with known or suspected of having activities against particular diseases can be used to establish molecular profiles and libraries for toxicity assessment. Antineoplastic drugs with similar toxicities, for example those listed in Table 1, can be used to compile molecular profiles by determining the alterations in gene or protein expression patterns in LSCs exposed to these drugs. Similarly, antibiotics with similar toxicities can also be assessed for their alterations in gene or protein expression patterns in LSCs. Also used are drugs controlling diabetes, drugs for lowering lipid levels, or anti-inflammatory drugs. Once a composite library comprising molecular profiles of specific type of drugs having similar toxicities is established, it can be used to screen for new drug leads of the similar type for their potential toxicities. Again, the experimental procedures as described above in general, and in more detail in the following examples, are followed for compiling molecular profiles and libraries, and for typing/ranking toxicities of new drug leads.

### Example 4

### Example 2 from U.S. patent application Publ. 20020012905

Establishing Protein Profiles for Chemical Agents Relating to Tissue/Organ Toxicities

This Example demonstrates the culturing of liver stem cells, the exposure of the liver stem cells to different chemical agents having pre-determined tissue or organ toxicities, and the determination of changes in protein expression in the liver stem cells.

Isolation of Cells

Human liver progenitor cells are isolated, purified and culture-expanded according to methods described below:

Method 1

Liver precursor cells are isolated according to Reid et al., U.S. Pat. Nos. 5,576,207 and 5,789,246. Briefly, a liver section is placed in an ice-cold PBS solution that contain 500-5000 mg/L glucose, and 2-10% antibiotics (e.g., penicillin and streptomycin). The liver section is minced and sequentially digested with a solution containing collagenase, pronase and deoxyribonuclease, prepared in a saline solution containing 1 mM CaCl₂. Digestion is done at 37° C. in a shaking water bath for about 20 minutes. The partially digested tissue is then strained through a tissue sieve by gravity and the undigested remnants are re-digested two times. Collected cells are then washed with saline solution. Cells are plated on or in a matrix of collagen Type IV so as to allow them to polarize and feed through a basal surface such as a Millicell support. Matrix-bound cells are provided with an embryonic liver-derived stromal cell feeder layer, and cultured in a basal medium (e.g., RPMI 1640, Ham's F10, Ham's F12) containing less than 0.4 mM calcium and 10 μg/ml insulin, 10 μU/ml growth hormone, 20 mU/ml prolactin, 10 μg/ml glucagon, 50 ng/ml EGF, 10⁻⁸M dexamethasone, 10⁻⁹MT3, 3×10⁻¹ M selenium, 10⁻⁷M copper and 10⁻¹⁰M zinc (medium containing the foregoing is hereinafter referred to as “medium A”). Alternatively, the medium further contains 0.4% bovine serum albumin, 76 mEq per liter of free fatty acid mixture having 31% palmitic acid, 2.8% palmitoleic acid, 11.6% stearic acid, 13.4% oleic acid, 35.6% linoleic acid and 5.6% linolenic acid (medium containing the foregoing is hereinafter referred to as “medium B”). Medium A or B containing 10⁻⁶ M dexamathasone, 0.1 to 100 μg/ml insulin, 50 ng/ml multi-stimulating activity (Sigma Chemical Co. St. Louis, Mo.), 25 to 100 ng/ml EGF, 10⁻⁴M norepinephrine, 25 μl/ml hepatopoietins and 10 ng/ml FGF's, can also be used. Liver precursor cells obtained by this method are analyzed according to the present invention as described below.

Method 2

Hepatoblasts are isolated according to the methods described in Reid et al., U.S. Pat. No. 6,069,005. Briefly, livers are dissected from rat fetuses of gestational day 14, and placed in fresh ice-cold Hank's Salt Solution (HBSS). After all tissues are collected and non-hepatic tissue removed, HBSS-5 mM EGTA is added to a final EGTA concentration of 1 mM. Livers are then gently triturated 6 to 8 times to partially desegregate the tissue and then centrifuged at 400 g for 5 minutes at 4° C. All subsequent centrifugation steps are performed at the same settings. Supernatant is removed and the pellet of cells and tissue resuspended in 0.6% collagenase D (Boehringer Mannheim, Indianapolis, Ind.) in HBSS containing 1 mM CaCl₂, gently triturated and then stirred at 37° C. for 15 minutes. The dispersed cells are pooled, suspended in HBSS containing 1 mM EGTA and filtered through a 46 μm tissue collector (Bellco Glass, Inc., Vineland, N.Y.). The cell suspension is centrifuged and cells resuspended in HBSS supplemented with MEM amino acids, MEM vitamins, MEM non-essential amino acids, insulin (10 μg/ml), iron-saturated transferrin (10 μg/ml), free fatty acids (7.6 mEq/L), trace elements, albumin (0.1%, fraction V, fatty acid free, Miles Inc., Kankakee, Ill.), myo-inositol (0.5 mM) and gentamicin (10 μg/ml, Gibco BRL, Grand Island, N.Y.) (HBSS-MEM). Cell number and viability are determined by hemacytometer and trypan blue exclusion.

To remove erythroid cells, panning dishes prepared according to standard procedure with a rabbit anti-rat RBC IgG (Rockland Inc., Gilbertsville, Pa.) are used. Antibodies (0.5 mg/dish) diluted in 0.05M Tris pH 9.5 are poured on 100 mm² bacteriological polystyrene petri dishes (Falcon, Lincoln Park, N.J.). The dishes are swirled to evenly coat the surface and incubated at room temperature for about 40 minutes. Coated dishes are washed four times with PBS and once with HBSS containing 0.1% BSA prior to use.

Cell suspension containing up to 3×10⁷ cells are incubated at 4° C. for about 10 minutes in the dishes coated with the rabbit anti-rat RBC IgG. Non-adherent cells are removed by aspiration and the plates washed three times with HBSS-0.1% BSA-0.2 mM EGTA and centrifuged. The cell pellet is resuspended in HBSS-MEM, and RBC panning repeated. Following the second RBC panning, cell number and viability are determined again.

Cells recovered after RBC panning are then labeled in suspension by incubating with mouse monoclonal antibody OX-43 ( 1/200=15 μg/ml, MCA 276, Bioproducts for Science, Indianapolis, Ind.) and monoclonal antibody 374.3 ( 1/500- 1/750, available from R. Faris and D. Hixon, Brown University, Providence, R.I.) simultaneously at 4° C. for 40 minutes. Secondary antibodies are PE-conjugated anti-mouse IgG, heavy chain specific (Southern Biotechnology Inc., Ala.) and FITC-conjugated anti-mouse IgM, heavy chain specific (Sigma Chemical Co., St. Louis, Mo.).

Cells before and after sorting are maintained at 4° C. and in HBSS-MEM. After antibody labeling, propidium iodide at 10 μg/ml is added to each sample. Fluorescence activated cell sorting is performed with a Becton Dickinson FACSTAR+′ (San Jose, Calif.) using a 4 W argon laser with 60 mW of power and a 100 μm nozzle. Fluorescent emission at 488 nm excitation is collected after passing through a 530/30 nm band pass filter for FITC and 585/42 nm for PE. Fluorescence measurements are performed using logarithmic amplification on biparametric plots of FL1 (FITC) vs. FL2 (PE). Cells are considered positive when fluorescence is greater than 95% of the negative control cells.

For measurement of physical characteristics of cells, FACSTAR+parameters are FSC gain 8 and SSC gain 8. HBSS is utilized as sheath fluid. List mode data are obtained and analyzed using LysisII software.

To determine positivity to a single antibody dot plots of fluorescence vs. side scatter are used. Density plots FL1 vs. FL2 are used to select populations with respect to expression of both antigens. A sort enhancement module is used for non-rectangular gating and use of multiparametric gating to select populations of interest.

Sorted cells from all populations are plated in a serum-free, hormonally-defined medium with .alpha.-MEM as the basal medium to which the following components are added: insulin (10 μg/ml); EGF (0.01 μg/ml, Upstate Biotechnology, Lake Placid, N.Y.); growth hormone (10 μU/ml); prolactin (20 mU/ml); Triiodothyronine (10⁻⁷ M); dexamethasone (10⁻⁷ M); iron saturated transferrin (10 μg/ml); folinic acid (10⁻⁸ M, Gibco BRL, Grand Island, N.Y.), free fatty acid mixture (7.6 mEq/L; Nu-Chek-Prep, Elysian, Minn.); putrescine (0.02 μg/ml); hypoxanthine (0.24 μg/ml); thymidine (0.07 μg/ml); bovine albumin (0.1%, fraction V, fatty acid free, Miles Inc. Kankakee, Ill.); trace elements; CuSO₄.5H₂O (0.0000025 mg/l), FeSO₄.7H₂O (0.8 μmg/l), MnSO₄.7H₂O (0.0000024 mg/l), (NH₄)₆Mo₇O₂₀.2H₂O (0.0012 mg/l), NiCl₂.6H₂O (0.000012 mg/l), NH₄VO₃ (0.000058 mg/l), H₂SeO₃ (0.00039 mg/l); Hepes (31 mM) and Gentamicin (10 μg/ml, Gibco BRL, Grand Island, N.Y.) [HDM]. Reagents are supplied by Sigma Chemical Company, St. Louis, Mo., unless otherwise specified. The trace element mix is available from Dr. I. Lemishka, Princeton University, N.J.

Aliquots of each cultured population as well as cytospins of various cell suspensions are fixed with ice-cold ethanol or acetone. After blocking with PBS containing 1% BSA for 30 minutes at room temperature, the fixed cells are studied by indirect immunofluorescence using the following primary antibodies: polyclonal rabbit-anti-rat albumin (United States Biochemical Corporation, Cleveland, Ohio), rabbit-anti-mouse AFP antiserum (ICN Biomedical, In., Costa Mesa, Calif.), monoclonal mouse-anti-human cytokeratin 19 (Amersham Life Science, Arlington Heights, Ill.), polyclonal rabbit-anti-human IGF II receptor (Dr. Michael Czech, University of Worchester, Mass.), mouse monoclonal anti-rat-Thy-1 (OX-7, Bioproducts for Science, Indianapolis, Ind.), monoclonal mouse-anti-desmin (Boehringer Mannheim, Indianapolis, Ind.), and 258.26, a monoclonal mouse-anti-rat antibody identifying postnatal hepatocytes as well as some fetal liver parenchymal cells (Drs. R. Faris and D. Hixon, Brown University, R.I.). Second antibodies include species specific Rhodamine conjugated antibodies corresponding to the primary antibodies. Negative controls consist of cells stained with mouse or rabbit IgG or mouse isotype controls. Freshly isolated adult hepatocytes are used as positive controls for albumin staining. Gamma-glutamyltranspeptidase (GGT) is assayed by immunochemistry on ethanol fixed cells using the method described by Rutenberg et al., J. Hist. Cyt. (1969), Vol. 17, pp. 517-526.

The cell population containing primarily immature, progenitor liver cells is identified as the population expressing albumin, alpha-fetoprotein and GGT. In some instances, the identity of the cells are confirmed by Northern and/or Western blot analysis for markers of these cells, which are described herein and known in the art.

The liver progenitor cells obtained are maintained in HDM and analyzed according to the present invention as described below.

Method 3

Livers are dissected from rat fetuses at day 15 of gestation and placed into ice-cold, Ca²⁺ free HBSS containing 0.8 mM MgCl₂, 20 mM HEPES, pH7.3 and gently agitated at room temperature for about 1 minute. After removal of non-hepatic tissue, livers are gently triturated and then stirred at 37° C. for about 10-15 minutes with 0.6% type IV collagenase (Sigma Chemical Co., Lot 11 H6830, St. Louis, Mo.) in HBSS containing 1 mM CaCl₂ and 0.06% DNAse I (Boehringer Mannheim, Indianapolis, Ind.). At 5 minute intervals, tissue fragments are allowed to sediment at 1 g. Supernatant is recovered and fresh collagenase solution added. The dispersed cells are pooled, suspended in HBSS containing 5 mM EGTA and filtered through a 46 μm tissue collector (Bellco Glass, Inc., Vineland, N.Y.) under 1 g. The resultant cell suspension is centrifuged at 4° C. for about 5 minutes under 450 g. The cell pellet is resuspended in HBSS containing 0.2 mM EGTA and 0.5% BSA (HBSS-EGTA-0.5% BSA), and the cell number is estimated. Cell viability is assessed by exclusion of 0.04% trypan blue, and an aliquot of the suspension is centrifuged at 450 g for about 5 minutes.

To immunoadhere hemopoietic and endothelial cells onto antibody-coated polystyrene dishes, panning dishes are prepared according to standard procedures with rabbit anti-rat RBC IgG (Inter-cell Technologies, Inc., Hopewell, N.J.) and goat IgG directed towards mouse whole IgG molecule (M-3014, Sigma, St. Louis, Mo.). Antibodies (0.5 mg/dish) diluted in 0.05M Tris pH 9.5 are poured on 100 mm² bacteriological polystyrene petri dishes (Flacon, Lincoln Park, N.J.) to evenly coat the surface and incubated at room temperature for about 40 minutes. Coated dishes are washed with PBS and then HBSS containing 0.1% BSA prior to use.

Cell suspension containing up to 3×10⁷ cells is incubated at 4° C. for 10 minutes in the coated dishes. Supernatant containing non-adherent cells is removed by gentle aspiration while tilting and swirling, combined with washes of HBSS-EGTA-0.1% BSA, and centrifuged at 4° C. for about 5 minutes under 450 g. Cells are pooled and repanned with a fresh dish coated with rabbit anti-rat RBS IgG. Non-adherent cells are then removed as above and resuspended with HBSS-EGTA-0.5% BSA to a concentration of 1×10⁷/ml. The enriched hepatoblasts are then incubated simultaneously at 4° C. for 40 minutes with mouse monoclonal antibody OX-43 (15 μg/ml, MCA 276, Serotec, Indianapolis, Ind.). After washing, enrichment for hepatoblasts is achieved by panning cells at 4° C. for 10 minutes in a dish coated with the goat anti-mouse whole IgG antibody. Non-adherent cells are removed as above.

The liver progenitor cells obtained are maintained in HDM (see method 3 above) and analyzed according to the present invention as described below.

Method 4

Liver progenitor cells are isolated according to the methods described in Naughton et al., U.S. Pat. No. 5,559,022. Briefly, liver is removed from adult rats according to standard procedures. The liver is placed on a modified Buchner funner and perfused with a buffer containing Ca²⁺ and 0.05 g/dl type IV collagenase (Sigma Chemical Co., MO) in a recirculating system for about 15-20 min. The liver is then transferred to a Petri dish containing collagenase buffer supplemented with 1.5% BSA and the hepatocytes are liberated into suspension after the perforation of Glisson's capsule, filtered through a 185 μm nylon sieve, pelleted by centrifugation, and resuspended in complete medium, DMEM conditioned with 6% fetal bovine serum and 10% equine serum and supplemented with 35 μl glucagon (Sigma #G9261), 10 μg insulin (Sigma #14011), 0.25 g glucose, and 250 μl hydrocortisone hemisuccinate per 500 ml of medium. Hepatic cells are separated into subpopulations using Percoll gradient centrifugation.

To obtain liver progenitor cells, a population of large (about 30 μm in diameter), acidophilic cells which proliferate and differentiate in culture to cells resembling mature hepatocytes is separated as follows: single cell suspensions of freshly isolated liver cells are centrifuged (500×g/5 min) and the pellet is resuspended in medium. The cell suspension is layered over 25 ml of a 70% v/v solution of ‘neat’ Percoll and 1×PBS and centrifuged at 800×g for 10 min. The two lower zones (of 4) are pooled, washed, and centrifuged against 25%1/50% (v/v/,neat Percoll/1×PBS) discontinuous gradient yielding a distinct interface zone and a pellet. The interface (density=1.0381 g/ml) consists of about 90% large, lightly acidophilic, mono- or binuclear cells with multiple, prominent nucleoli. Liver precursor cells obtained by this method are then analyzed according to the present invention as described below.

Method 5

Liver progenitor cells are isolated according to the methods described in Faris, PCT Publication WO00/03001. Briefly, liver is excised, minced and placed in a suspension buffer containing HBSS with 0.1 M Hepes. The minced tissue is incubated at 37° C. on a stirring plate for about 40-50 minutes. The combined suspension is sequentially filtered through a 230 micron steel mesh filter, and a 60 micron nylon mesh filter. Remnants remaining on the filters are washed off and placed in digestion buffer, which is a CMF media (Gibco) solution containing 0.02 g of bovine serum albumin, 0.1M Hepes, CaCl₂ (500 mM), STI (0.025 g/100 ml; Gibco), and collagenase Type IV (60 units/ml), @ pH 7.4-7.5. Cells are incubated at 37° C. in a shaker water bath. After 20 minutes, the cell suspension is removed to a tube and allowed to settle by gravity.

The supernatant and the remnant (settled material) are then separated. The supernatant is decanted and centrifuged at 80×g for 5 minutes. Fresh digestion buffer is added to the cells and the cells placed back in the shaking water bath. The pellet remaining after the centrifugation is resuspended with washing buffer (DMEM-F12 and BSA (1 g/100 ml), @ pH 7.2-7.3.

The cell suspension is filtered through a 60 micron nylon mesh filter and then mixed with an equal volume of 90% Percoll and 10% 10×DMEM-F12. This is centrifuged at 300×g for about 5 minutes. The pellet is resuspended in washing buffer (as described above), and centrifuged at 120×g for about 5 minutes. The pellet is then resuspended in washing buffer.

Dynabeads conjugated to a mouse monoclonal antibody specific for rat bile duct and mesothelial cells (IgG_{2b}) are added to the cell suspension, and incubated at 4° C. on a rotator for about 10 minutes. The suspension is then placed on a magnet to remove antibody-positive cells, which are discarded. This step is repeated at least 3 more times. The antibody-negative cells are subjected to more incubations with Dynabeads conjugated to an antibody specific for CCAM (e.g., anti-rat cell-CAM 105; Endogen), and antibody-positive cells with a stem cell attached (e.g., cell clusters such as doublets and triplets) are cultured and cytospinned.

Isolated cell clusters are trypsinized to dissociate the cell clusters, then exposed to antibodies specific for cell markers such as CK19 (Amersham), CCAM (Endogen), dipeptidyl peptidase-4 (Endogen) in combination with magnetic beads or FACS sorting to enrich for the stem cells.

Liver precursor cells obtained by this method are analyzed according to the present invention as described below.

Exposure of Cells to Test Chemical Composition and Methods of Analysis of Protein Expression

Cells isolated as described above are plated at high density (e.g., 50,000 to 100,000 cells/cm² per well) in wells coated with type I collagen extracted from rat tail tendon, to allow differentiation of cells. A drug with pre-determined toxicity, such as troglitazone, which is a drug designed for the control of diabetes which has shown rare but severe liver toxicity and recently removed from the market, is added at a final concentration of about 20 μM to one group of plates (group “A”) containing the LSCs. On the same day, another drug with pre-determined toxicity, such as erythromycin estolate (Sigma, catalog number E8630), which is a form of erythromycin with known liver toxicity, is added to a second group of plates (group “B”) at a final concentration of about 50 μM. A third group of plates containing the cultured cells (group “C1”) is cultured without any added drugs to serve as a control. Additionally, plates containing only tissue culture medium (group “C2”) are cultured alongside those containing cultured cells as a control for degradation of proteins in the culture medium. Following a period of exposure of the cells to the drugs, for example after about ten, twenty, thirty and forty days, the cultures are harvested, the cells washed with a buffer such as PBS, and then lysed in a buffer that contains, for example, PBS, 0.5% Triton X-100 for about 10 minutes on ice. The nuclei are pelleted, and the supernatant removed and stored at −80° C. until analysis. The nuclei are lysed in a buffer such as PBS with 0.2% SDS and dounce homogenized to shear the DNA. The insoluble material is pelleted and the nuclear lysates stored at −80° C. until analyzed. Cytoplasmic and nuclear lysates are also taken on day zero prior to exposure to any test chemical compositions to serve as additional controls.

The lysates and medium samples are diluted by, for example, 3 fold in buffer containing 50 mM Tris-HCl at pH 8, and 0.4 M NaCl. Aliquoted samples of diluted lysate or medium are placed in a sizing spin column that fractionates the sample with a size cut-off of, for example, 30 kD and equilibrated in 50 mM Tris-HCl, pH 8 and 50 mM NaCl. The column is spun at an appropriate force and for an appropriate period, such as 700 g for 3 minutes, for each fraction. Multiple fractions of about 25 μL are collected for each column using the column equilibrated buffer.

The samples are partitioned by surface enhanced laser desorption/ionization (“SELDI”), and proteins are detected by mass spectroscopy. SELDI permits proteins to be captured on a surface of choice, which can then be washed at selected stringency, to permit fractionation according to desired characteristics such as affinity for metal ions of the surface used for capture.

Ciphergen normal phase chips (Ciphergen Biosystems, Palo Alto, Calif.) are used to partition the proteins in the fractions generated by the spin columns. Aliquots of about 1 μl of each fraction are deposited on a spot on the chip, and the sample is air dried at room temperature for about 5 minutes. A mixture of about 0.5 μL of saturated sinapinic acid (“SPA”) in 50% acetonitrile with 0.5% trifluroacetic acid (“TFA”) is applied to each spot. The chip is again permitted to air dry for about 5 minutes at room temperature, and a second aliquot of the SPA mixture is applied.

Chips are read by the Ciphergen Protein Biology System 1 reader. Exemplary reader settings are as follows. Auto mode is used for data collection, at the SELDI quantitation setting. Two sets of protein profiles are collected, one at low laser intensity (at 15 with filter out) and one at high laser intensity (at 50 with filter out), detector set at 10. An average of 15 shots per location on the same sample spot are made. Protein profiles from different lysates are compared using SELDI software (Ciphergen Biosystems, Palo Alto, Calif.). This program assumes two proteins with a molecular weight within about 1% of each other are the same. It then quantitates the results, compares the test samples against the control samples, and prints a graph showing the amount of each protein in the control as a horizontal line, with any reduction or excess in the amount of each protein in the test sample compared to the amount of that protein in the control sample as a line below or above the line representing the control.

### Example 5

### Example 3 from U.S. patent application Publ. 20020012905

Screening of Anti-Cancer Drugs for Tissue and Organ Toxicities

This example illustrates using the LSC system for screening anti-cancer agents for their tissue or organ toxicities.

Compounds and drugs (both anti-cancer and therapeutic) that have known toxicities and biology endpoints in humans and/or animals are selected for compiling their gene or protein expression profiles in LSCs. In addition, compounds are selected with related known mechanisms of activities and with regard to compounds that have been used in previous studies to correlate clinical outcomes with human in vitro cell culture effects. Table 3 of the cited publication.

a. Establishing Gene Expression Profiles

The gene expression pattern of a selected compound is measured and quantified using cDNA microarrays and is normalized with cellular differentiation. The gene expression pattern of the compound is compared with a control LSC culture not exposed to the compound or, where appropriate, LSC cultures treated with related drugs with similar function or dose limiting toxicity. By compiling the gene expression profiles for a number of anti-cancer agents having similar or related toxicities, common alterations in gene expression are discerned and correlated with the toxicities, and are used as surrogate profiles for assessing the toxicities of test anti-cancer drug candidates.

The cDNA microarray can be any one of many kinds that are known and available in the art, for example, as described in Shalon et al (1996), Genome Res 6:639-645. cDNA microarrays allow for the simultaneous monitoring of the expression of thousands of genes, by direct comparison of control and chemically-treated cells. 3′ expressed sequence tags (ESTs) are arrayed and spotted onto glass microscope slides at a density of hundreds to thousands per slide using high speed robotics. Fluorescent cDNA probes are generated from control and test RNAs using a reverse transcriptase reaction with labeled dUTP using fluors that excite at two different wavelengths, i.e. Cy3 and Cy5, which allows for the hybridization of both the control and test RNA to the same chip for direct comparison of relative gene expression in each sample. The fluorescent signal is detected using a specially engineered scanning confocal microscope. A collection of 15,000 sequence verified human clones and 8700 mouse clones can be used in making cDNA microarrays. These microarrays are ideal for the analysis of gene expression patterns in LSC cultures treated with a variety of agents.

Another example of microarray analysis is described in Lockhart et al., U.S. Pat. No. 6,040,138. In this method, labeled RNA or cDNA from target cells are hybridized to a high density array of oligonucleotide probes where the high density array contains oligonucleotide probes complementary to subsequences of target nucleic acids in the RNA or cDNA sample. 20 mer oligonucleotide probes prepared as described in Lockhart et al., supra, are arrayed on a planar glass slide. Labeled RNAs are generated from control and test LSCs using methods known in the art, such as incubating cells in the presence of labeled nucleotides. Alternatively, labeled cDNAs are prepared from RNAs of the test and control cells using a reverse transcription reaction with labeled nucleotides, such as dUTP using fluors that excite at different wavelengths. Signal from the labeled RNA or cDNA can be read by a laser-illuminated scanning confocal fluorescence microscope. The microarray in this method is capable of simultaneous monitoring of more than 10,000 different genes.

Briefly, RNAs are isolated from control and treated LSCs. Total RNA are prepared using the RNAeasy kit from Qiagen. Subsequently, RNA are labeled either with Cy3 or Cy5 dUTP in a single round of reverse transcription. The resultant labeled cDNAs are mixed in a concentrated volume and hybridized to the arrays. Hybridizations are incubated overnight at 65° C. in a custom designed chamber that prevents evaporation. Following hybridization, the chip is scanned with a custom confocal laser scanner that will provide an output of the intensity of each spot in the array for both the Cy3 and Cy5 channels. The data are then analyzed with a software package that contains additional extensions. These extensions allow for the integration of a signal across each spot, normalization of the data to a panel of designated housekeeping genes, and statistical calculations to generate a list of genes whose ratios are outliers, or significantly changed by the treatment. In addition to the image analysis software, informatics packages such as Spot-Fire and GeneSpring, both of which are commercially available, are used to allow clustering and analysis of genes in multiple experiments across dose and/or time. cDNA microarray technology, in general, is still being validated as a viable technique for providing quantitative data. While the ratio of red/green provides good qualitative data on the relative level of expression of a gene in one population versus the other, it is not an absolute value of the level of induction/down regulation of that gene. Each pair of samples on the arrays is hybridized in triplicate. Outliers that are consistently induced or suppressed in two of the three hybridization experiments are further validated by a traditional RNA quantitation method, such as Northern blot or RT-PCR.

Each drug is tested at least three times on separate LSC cultures for its effects on growth, differentiation and RNA expression. Cell counts (growth), amount of cells expressing/not expressing and/or exhibiting a particular differentiation marker/characteristic (differentiation) and RNA levels/cDNA microarray data (RNA expression) are averaged for the three or more experiments and the mean and SEM determined. All results are normalized using approximately 15 “house keeping” genes. This allows a quantitative comparison of the effects of the test drugs to control compounds that are not toxic in humans or animals. Statistical comparisons provide information for determining whether a given drug affects LSC gene expression compared to control drugs or non-treated cells and for determining whether a change in RNA in the cells is relevant.

b. Establishing Protein Expression Profiles

The protein expression profiles of the selected anti-cancer drugs are established using Ciphergen's SELDI mass spectroscopy (MS)-TOF system, as described in Example 2 of the cited publication (above). Total cell lysates from harvested LSC cultures are prepared in either 0.1% SDS or Triton-X100 (0.5%) and an equal protein mass is directly applied to protein array chips using manufacturer's protocols. For some situations it may be desirable to add a defined mass of one or more known control peptides as internal calibration and quantification standards to allow more quantitative comparisons between chips and samples. Each chip can analyze two drugs in triplicate. After working out the stringency conditions and experimental replications, on average 6 ProteinChips™ per test compound are used.

The Ciphergen technology allows for the proteins in the sample to be captured, retained and purified directly on the chip. The proteins on the microchip are then analyzed by SELDI. This analysis determines the molecular weight of proteins in the sample. An automatic readout of the molecular weights of the purified proteins in the sample can then be assessed. Typically this system has a CV of less than 20%. The Ciphergen data analysis system normalizes the data to internal reference standards and subtracts the readout of proteins found in control cells from those in drug treated cells. This data analysis reveals protein expression stimulated by the drugs as well as proteins only found in the control cells whose expression is inhibited by the drug. The analysis provides a qualitative readout of protein expression between a control and treated group. Analysis of multiple samples provides an average fold change in protein expression and a relative measure of variability. This can be represented as a mean.+−.SEM which can provide a statistical measure of the protein changes. This analysis is used to determine whether drugs that induce similar forms of toxicity in humans cause similar changes in protein expression in LSCs. Each drug is analyzed on at least 3 separate groups of LSCs.

### Example 6

### Example 1 from U.S. patent application Publ. 2006/0029574

Differentially Expressed Proteins in Gleevec-Sensitive and Gleevec-Resistant CML Resolved by 2-D Gel Electrophoresis

Bone marrow aspirate samples from 8 Gleevec sensitive and 6 Gleevec resistant CML patients were analyzed for differential protein expression using 2-D gel electrophoresis.

Two-Dimensional Gel Electrophoresis. Bone marrow aspirate samples were subjected to ProteEx protocol for protein purification and quantitative assay (U.S. patent application Ser. No. 10/301,512, incorporated by reference). Protein separation was conducted as mentioned before (Kuncewicz et al., 2003). Briefly, the purified proteins were suspended in a buffer containing 8 M urea, 2 M thiourea, 1% Triton X-100, 1% DTT, and 1% ampholytes pH 3-10. An aliquot of 100 μg of protein was loaded onto an 11 cm IEF strip (Bio-Rad Laboratories, Hercules Calif.), pH 4-7 and 6-11. Focusing was conducted on IEF cells (Bio-Rad Laboratories) at 250 V for 20 minutes followed by a linear increase to 8000 V for 2 hours. The focusing was terminated at 20,000 volt-hours.

Strips were then equilibrated in 375 mM Tris buffer, pH 8.8, containing 6 M urea, 20% glycerol, and 2% SDS. Fresh DTT was added to the buffer at a concentration of 30 mg/ml and incubated for 15 minutes, followed by an additional 15-minute incubation with fresh buffer containing 40 mg/ml iodoacetamide. Strips were then loaded onto the second dimension using Criterion pre-cast gradient gels (Bio-Rad Laboratories) with an acrylamide gradient of 10-20%. Gels were then stained using SyproRuby fluorescent dye.

Gel Image Analysis. Stained gels were scanned on laser scan Molecular imager FX (Bio-Rad Laboratories). The results of digital fluorescent image analysis of gel images from Gleevec-sensitive samples were compared to Gleevec-resistant samples by qualitative and quantitative comparison of protein patterns using pre-mixed internal protein standards (BioRad Laboratories) as landmarks.

Spot density was quantitatively normalized based on the density of each spot versus the total density of all detected spots. The software was set up for analysis of PPM for each spot and also for highlighting fold differences between spots in any set of image comparison. A reproducible density difference was considered significant with a coefficient of variation of <20%.

Tryptic Digestion, MALDI-TOF MS, and Peptide Mass Fingerprinting Analysis. Following differential expression analysis of the proteins, spots of interest were excised from the gel using the ProteomeWorks robotic spot cutter (Bio-Rad Laboratories). Excised spots were robotically in-gel digested on a MultiPROBE II (Packard, Downers Grove, Ill.) as follows: gel spots were washed twice in 100 mM NH₄HCO₃ buffer, followed by soaking in 100% acetonitrile for 5 minutes, aspiration of the acetonitrile, and drying of the gels for 30 minutes.

Re-hydration of the gels using 20 μg/ml trypsin (Promega, Madison, Wis.) suspended in 25 mM NH₄HCO₃ buffer was followed by incubation at 37° C. for 14-20 hours. The digested peptides were extracted twice using a solution of 50% acetonitrile and 5% trifluoroacetic acid for 40 minutes. Peptide extracts were desalted and concentrated using reverse phase C18 Zip-tips (Millipore, Bedford, Mass.) and robotically placed on MALDI chips using the SymBiot I (Applied Biosystems, Foster City, Calif.).

Mass spectral analyses were conducted on MALDI-TOF Voyager DE PRO (Applied Biosystems). Spectra were carefully scrutinized for acceptable signal-to-noise ratio (S/N) to eliminate spurious artifact peaks from the peptide molecular weight lists and both internal and external standards were employed. Corrected lists were subjected to database searches using both the NCBI and Swiss protein data banks with a minimum matching peptide setting of 4, mass tolerance settings of 50-250 ppm, and for a single trypsin miss-cut.

Results. A total of 19 spots were found to be differentially expressed between Gleevec-sensitive samples and Gleevec-resistant samples. In the pI 4-7 range, 5 spots were consistently up-regulated (spots 2319, 2414, 2417, 2418, and 2421) and 2 spots were consistently down-regulated (spots 7406 and 7524) in samples from Gleevec-sensitive patients relative to samples from Gleevec-resistant patients (FIGS. 2A, 2B, 2C, and 2D). In the pI 6-11 range, 12 spots were consistently up-regulated in the samples from the Gleevec-sensitive patients relative to samples from Gleevec-resistant patients (FIGS. 3A, 3B, 3C, and 3D). The differentially expressed spots were excised and the proteins identified, as described above. The proteins up-regulated in Gleevec-sensitive cells are listed in Table 4 in the cited publication, and the proteins down-regulated in Gleevec-sensitive cells are listed in Table 5 in the cited publication.

### Example 7

### From U.S. Pat. No. 6,670,194

Quantitative Analysis of Protein Expression in Different Cell States

The protein reactive affinity reagent strategy was applied to study differences in steady-state protein expression in the yeast, S. cerevisiae, in two non-glucose repressed states (Table 3 in the cited reference). Cells were harvested from yeast growing in log-phase utilizing either 2% galactose or 2% ethanol as the carbon source. One-hundred μg of soluble yeast protein from each cell state were labeled independently with the isotopically different affinity tagged reagents. The labeled samples were combined and subjected to the strategy described in Scheme 1. One fiftieth (the equivalent of approximately 2 μg of protein from each cell state) of the sample was analyzed.

Glucose repression causes large numbers of proteins with metabolic functions significant to growth on other carbon sources to be minimally expressed (Ronne, H. (1995), “Glucose repression in fungi,” Trends Genet. 11:12-17. Scriver, C. R. et al. (1995), The Metabolic and Molecular Bases of Inherited Disease, McGraw-Hill, N.Y.; Hodges, P. E. et al. (1999), “The Yeast Proteome Database (YPD): a model for the organization and presentation of genome-wide functional data,” Nucl. Acids Res. 27:69-73.). Growth on galactose or ethanol with no glucose present results in the expression of glucose repressed genes. Table 3 in the cited reference presents a selection of 34 yeast genes encountered in the analysis, but it contains every known glucose-repressed genes that was identified (Mann, M., and Wilm, M. (1994), “Error-tolerant identification of peptides in sequence databases by peptide sequence tags,” Anal. Chem. 66:4390-4399). Each of these genes would have been minimally expressed in yeast grown on glucose. Genes specific to both growth on galactose (GAL1, GAL 10) as well as growth on ethanol (ADH2, ACH1) were detected and quantitated.

The quantitative nature of the method is apparent in the ability to accurately measure small changes in relative protein levels. Evidence of the accuracy of the measurements can be seen by the excellent agreement found by examining ratios for proteins for which multiple peptides were quantified. For example, the five peptides found from PCK1 had a mean ratio±95% confidence intervals of 1.57±0.15, and the percent error was <10%. In addition, the observed changes fit the expected changes from the literature (Ronne, H. 1995; Hodges, P. E. et al. (1999)). Finally, the observed changes are in agreement with the changes in staining intensity for these same proteins examined after two-dimensional gel electrophoresis (data not shown).

The alcohol dehydrogenase family of isozymes in yeast facilitates growth on either hexose sugars (ADH1) and ethanol (ADH2). The gene ADH2 encodes an enzyme that is both glucose- and galactose-repressed and permits a yeast cell to grow entirely on ethanol by converting it into acetaldehyde which enters the TCA cycle (FIG. 5A in the cited reference). In the presence of sugar, ADH1 performs the reverse reaction converting acetaldehyde into ethanol. The regulation of these isozymes is key to carbon utilization in yeast (Ronne, H. (1995)). The ability to accurately measure differences in gene expression across families of isozymes is sometimes difficult using cDNA array techniques because of cross hybridization (DeRisi, J. L. et al. (1997), “Exploring the metabolic and genetic control of gene expression on a genomic scale,” Science 278:680-6.). The method of this invention applied as illustrated in Scheme 1 succeeded in measuring gene expression for each isozyme even though ADH1 and ADH2 share 93% amino acid (88% nucleotide) sequence similarity. This was because the affinity tagged peptides from each isozyme differed by a single amino acid residue (valine to threonine) which shifted the retention time by more than 2 min and the mass by 2 daltons for the ADH2 peptides (FIG. 5B in the cited reference). ADH1 was expressed at approximately 2-fold high levels when galactose was the carbon source compared with ethanol. Ethanol-induction of ADH2 expression resulted in more than 200-fold increases compared with galactose-induction.

The results described above illustrate that the method of this invention provides quantitative analysis of protein mixtures and the identification of the protein components therein in a single, automated operation.

The method as applied using a sulfhydryl reactive reagent significantly reduces the complexity of the peptide mixtures because affinity tagged cysteine-containing peptides are selectively isolated. For example, a theoretical tryptic digest of the entire yeast proteome (6113 proteins) produces 344,855 peptides, but only 30,619 of these peptides contain a cysteinyl residue. Thus, the complexity of the mixture is reduced, while protein quantitation and identification are still achieved. The chemical reaction of the sulfhydryl reagent with protein can be performed in the presence of urea, sodium dodecyl sulfate (SDS), salts and other chemicals that do not contain a reactive thiol group. Therefore, proteins can be kept in solution with powerful stabilizing agents until they are enzymatically digested. The sensitivity of the μLC-MSⁿ system is dependent of the sample quality. In particular, commonly used protein solubilizing agents are poorly compatible or incompatible with MS. Affinity purification of the tagged peptides completely eliminates contaminants incompatible with MS. The quantitation and identification of low abundance proteins by conventional methods requires large amounts (milligrams) of starting protein lysate and involves some type of enrichment for these low abundance proteins. Assays described above, start with about 100 μg of protein and used no fractionation techniques. Of this, approximately 1/50 of the protein was analyzed in a single μLC-MSⁿ experiment. This system has a limit of detection of 10-20 fmol per peptide (Gygi, S. P. et al. (1999), “Protein analysis by mass spectrometry and sequence database searching: tools for cancer research in the post-genomic era,” Electrophoresis 20:310-319.). For this reason, in the assays described which employ μLC-MSⁿ only abundant proteins are detected. However, the methods of this invention are compatible with any biochemical, immunological or cell biological fractionation methods that reduce the mixture complexity and enrich for proteins of low abundance while quantitation is maintained. This method can be redundant in both quantitation and identification if multiple cysteines are detected. There is a dynamic range associated with the ability of the method to quantitate differences in expression levels of affinity tagged peptides which is dependent on both the intensity of the peaks corresponding the peptide pair (or set) and the overall mixture complexity. In addition, this dynamic range will be different for each type of mass spectrometer used. The ion trap was employed in assays described herein because of its ability to collect impressive amounts of sequencing information (thousands of proteins can potentially be identified) in a data-dependent fashion even though it offers a more limited dynamic quantitation range. The dynamic range of the ion trap (based on signal-to-noise ratios) varied depending on the signal intensity of the peptide pair and complexity of the mixture, but differences of up to 100-fold were generally detectable and even larger differences could be determined for more abundant peptides. In addition, protein expression level changes of more than 100-200-fold still identify those proteins as major potential contributors of the phenotypic differences between the two original cell states. The method can be extended to include reactivity toward other functional groups. A small percentage of proteins (8% for S. cerevisiae) contain no cysteinyl residues and are therefore missed by analysis using reagents with sulfhydryl group specificity (i.e., thiol group specificity). Affinity tagged reagents with specificities toward functional groups other than sulfhydryl groups will also make cysteine-free proteins susceptible to analysis.

The methods of this invention can be applied to analysis of low abundance proteins and classes of proteins with particular physico-chemical properties including poor solubility, large or small size and extreme pI values.

The prototypical application of the chemistry and method is the establishment of quantitative profiles of complex protein samples and ultimately total lysates of cells and tissues following the preferred method described above. In addition the reagents and methods of this invention have applications which go beyond the determination of protein expression profiles. Such applications include the following:

Application of amino-reactive or sulfhydryl-reactive, differentially isotopically labeled affinity tagged reagents for the quantitative analysis of proteins in immuno precipitated complexes. In the preferred version of this technique protein complexes from cells representing different states (e.g., different states of activation, different disease states, different states of differentiation) are precipitated with a specific reagent, preferably an antibody. The proteins in the precipitated complex are then derivatized and analyzed as above.

Application of amino-reactive, differentially isotopically labeled affinity tagged reagents to determine the sites of induced protein phosphorylation. In a preferred version of this method purified proteins (e.g., immunoprecipitated from cells under different stimulatory conditions) are fragmented and derivatized as described above. Phosphopeptides are identified in the resulting peptide mixture by fragmentation in the ion source of the ESI-MS instrument and their relative abundances are determined by comparing the ion signal intensities of the experimental sample with the intensity of an included, isotopically labeled standard.

Amino-reactive, differentially isotopically labeled affinity tagged reagents are used to identify the N-terminal ion series in MSⁿ spectra. In a preferred version of this application, the peptides to be analyzed are derivatized with a 50:50 mixture of an isotopically light and heavy reagent which is specific for amino groups. Fragmentation of the peptides by CID therefore produces two N-terminal ion series which differ in mass precisely by the mass differential of the reagent species used. This application dramatically reduces the difficulty in determining the amino acid sequence of the derivatized peptide.

### REFERENCES CITED

1. Link, 2-D proteome analysis protocols. Methods in Molecular Biology vol 112. Humana Press (1999).
2. Liebler, Introduction to proteomics. Tools for new biology. Human Press (2002).
3. Grandi, Genomics Proteomics and Vaccines. Wiley and Sons (2004).
4. Albala et al. Protein arrays, Biochips, and Proteomics. The next phase of gene discovery. Marcel Dekker (2003).
5. Fung, Protein arrays, methods and protocols. Methods in molecular biology vol. 264. Human Press (2004).
6. A trends guide to Proteomics. A supplement to Trends in Biotechnology 19(10) October 2001.
7. Arthur et al. Differential expression of proteins in renal cortex and medulla: A proteomic approach. Kidney International. 62: 1314-1321 (2002).
8. Han et al. Quantitative profiling of differentiation-induced microsomal proteins using isotope-coded affinity tags and mass spectrometry. Nature Biotechnology. 19: 946-951 (2001).
9. Gygi et al. Quantitative analysis of complex protein mixtures using isotope-coded affinity tags. Nature Biotech. 17: 994-999 (1999).
10. Greenbaum et al. Comparing protein abundance and mRNA expression levels on a genomic scale. Genome Biology. 4: 117-117.7 (2003).
11. U.S. Provisional patent application number U.S. 60/687,526. Methods for producing improved gene expression analysis comparison assay results.
12. Neidhardt et al. Physiology of the bacterial cell. A molecular approach. Chap 5. Sinauer Assoc. Inc. (1990).

For the purpose of explanation the foregoing discussion used specific nomenclature to provide a thorough understanding of the invention and its many embodiments. However, it will be apparent to one of skill in the art that this nomenclature and description are but one way to describe the invention and its mode of practice. Thus, the foregoing nomenclature and description are presented for the purpose of illustration and description, and they are not intended to be exhaustive or to limit the invention to the precise forms disclosed, and obviously many modifications and variations are possible as a result of the above techniques. The discussions presented were selected and described in order to best explain the present invention and its practical applications, and to thereby enable others skilled in the art to best practice the invention and various embodiments with various modifications, as are suited to the particular use contemplated.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually. However, the citation of any publication for its disclosure prior to the filing date should not be construed as an admission that the present invention is not entitled to antedate such publication by virtue of prior invention.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to GIVE EXAMPLES OF VARIATIONS THAT CAN BE MADE. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention.

Thus, additional embodiments are within the scope of the invention and within the following claims.

What is claimed is:

1. A method for producing improved particular protein (PP) expression analysis assay results (IR) for at least one cell sample, comprising
determining for each analyzed cell sample the number of cells or cell equivalents which are analyzed in the assay; and
normalizing the assay measured PP expression comparison results for the number of cell sample cells analyzed in the assay,
wherein said normalization produces assay results which are known to be improved in normalization and interpretability relative to such cell sample PP expression assay results obtained by prior art normalization practice.

2. The method of claim 1, wherein said PP is an endogenous protein.

3. The method of claim 1, wherein said PP is an exogenous protein.

4. The method of claim 1, wherein the PP expression result comprises one or more of:
a cell sample PP expression extent per cell result;
a cell sample PP abundance value;
a PP differential protein expression ratio (PP-DPER) for a cell sample comparison,
a protein expression profile (PEP) for a cell sample consisting of one or more PP relative or absolute PP abundance results, or
a differential protein expression profile(D-PEP) consisting of compared cell sample PEPs.

5. The method of claim 1, wherein said PP expression results comprises
a cell sample PP expression extent per cell result.

6. The method of claim 1, wherein said PP expression results comprises
a cell sample PP abundance value.

7. The method of claim 1, wherein said PP expression results comprises
a PP differential protein expression ratio (PP-DPER) for a cell sample comparison.

8. The method of claim 1, wherein said PP expression results comprises
a differential protein expression profile(D-PEP) consisting of compared cell sample PEPs.

9. The method of claim 1 wherein said cell sample comprises one or more of:
normal cells;
abnormal cells;
untreated cells;
treated cells;
physically treated cells;
chemically treated cells;
drug treated cells;
bioactive compound treated cells;
cells from a psychologically treated individual;
drug candidate treated cells;
toxic compound treated cells;
differentiated cells;
undifferentiated cells;
biological agent infected cells;
virus infected cells;
cells from an individual infected by a pathogenic bacterium;
cells from an individual infected by a eukaryotic microbe;
neoplastic cells;
cancer cells;
diseased cells;
pathological cells;
in vitro cultured cells;
in vitro cultured cells of an immortalized cell line;
in vivo sampled cells;
in vivo sampled cells of a particular tissue;
prokaryotic cells;
eukaryotic cells;
temporally treated cells;
mammalian cells;
mouse cells;
rat cells; and
human cells.

10. The method of claim 1, wherein said at least one cell sample comprises a plurality of cell samples.

11. The method of claim 10, wherein said at least one cell sample comprises at least 5 cell samples.

12. The method of claim 1, wherein said one or more particular protein(PP) improved results (IR) comprises IRs for a plurality of different PPs.

13. The method of claim 12, wherein said plurality of different PPs comprises at least 3 different PPs.

14. The method of claim 12, wherein said plurality of different PPs comprises at least 10 different PPs.

15. The method of claim 12, wherein said plurality of different PPs comprises at least 100 different PPs.

16. The method of claim 12, wherein said plurality of different PPs comprises at least 1000 different PPs.

17. The method of claim 8, wherein PPs comprising said cell sample PEP comprise at least one cellular protein.

18. The method of claim 17, wherein said cellular PP comprises a regulatory protein.

19. The method of claim 17, wherein said cellular PP comprises a membrane protein.

20. The method of claim 17, wherein said cellular PP comprises a protein from an infectious biologic agent.

21. The method of claim 17 wherein said cellular PP comprises a biomarker protein.

22. The method of claim 17, wherein said cellular PP comprises a drug or bioactive compound candidate or target PP.

23. The method of claim 17, wherein said cellular PP comprises a pathologic or disease related protein.

24. The method of claim 8, wherein said PEP is improved in quantitative accuracy, qualitative accuracy, or both, as compared to a PEP compiled from results which are not IRs.

25. The method of claim 8, wherein said PEP is improved in interpretability as compared to a PEP compiled from results which are not IRs.

26. The method of claim 8, wherein said PEP is improved in reproducibility as compared to a PEP compiled from results which are not IRs.

27. The method of claim 8, wherein said PEP is improved in intercomparability as compared to a PEP compiled from results which are not IRs.

28. The method of claim 8, wherein said PEP is improved in utility as compared to a PEP compiled from results which are not IRs.

29. The method of claim 1, wherein said determining one or more PP improved results (IR) for said cell sample is performed using a microarray assay.

30. The method of claim 1, wherein said determining one or more PP improved results (IR) for said cell sample is performed using a 2D gel electrophoresis assay.

31. The method of claim 1, wherein said determining one or more PP improved results (IR) for said cell sample is performed using at least one affinity binding media, method.

32. The method of claim 1, wherein said determining one or more PP improved results (IR) for said sample is performed using a mass spectroscopy method.

33. The method of claim 1, wherein said determining one or more PP improved results (IR) for said sample is performed using an immunoassay method.

34. The method of claim 1, wherein said determining one or more PP improved results (IR) for said cell sample is performed using an ELISA method.

35. A method for identifying a particular cell sample type of interest, comprising
comparing protein expression profiles (PEPs) incorporated improved results of at least one cell sample type of interest and at least one reference cell sample type of interest; and
identifying said cell sample type of interest based on best match comparison of the respective PEPs.

36. The method of claim 35, wherein said PEPs comprise expression results for a plurality of PPs.

37. The method of claim 35, further comprising
identifying for a plurality of PPs which PPs are differentially expressed in the cell sample of interest.

38. The method of claim 35, wherein said improved results are obtained by a method of any of claims 1-30.

39. The method of claim 35, further comprising
utilizing the method of any of claims 1-30 to determine a PEP for one or more cell samples of the said cell sample type of interest, or for one or more cell samples of a specified reference cell sample type of interest, or both; and
incorporating said results in at least one PEP.

40. The method of claim 35, wherein one or more regulated PPs are detectably expressed in both the cell sample of interest and said reference cell sample type.

41. The method of claim 35, wherein one or more down regulated PPs is not detectable as being expressed in one of the said cell samples.

42. The method of claim 35, wherein a said cell sample of interest or a said reference cell sample type or both comprises cells from one or more of:
normal cells;
abnormal cells;
untreated cells;
treated cells;
physically treated cells;
chemically treated cells;
drug treated cells;
bioactive compound treated cells;
cells from a psychologically treated individual;
drug candidate treated cells;
toxic compound treated cells;
differentiated cells;
undifferentiated cells;
biological agent infected cells;
virus infected cells;
cells from an individual infected by a pathogenic bacterium;
cells from an individual infected by a eukaryotic microbe;
neoplastic cells;
cancer cells;
diseased cells;
pathological cells;
in vitro cultured cells;
in vitro cultured cells of an immortalized cell line;
in vivo sampled cells;
in vivo sampled cells of a particular tissue;
prokaryotic cells;
eukaryotic cells;
temporally treated cells;
mammalian cells;
mouse cells;
rat cells; and
human cells.

43. The method of claim 35, wherein said cell sample type of interest comprises a plurality of separate different cell sample types.

44. The method of claim 35, wherein said reference cell sample type comprises a plurality of separate different cell sample types.

45. The method of claim 35, wherein said at least one cell sample type of interest or said reference cell sample type or both comprises at least 5 different cell sample types.

46. The method of claim 37, wherein said one or more PP improved results (IR) comprises IRs for a plurality of different PPs.

47. The method of claim 37, wherein said plurality of different PPs comprises at least 3 different PPs.

48. The method of claim 37, wherein said plurality of different PPs comprises at least 10 different PPs.

49. The method of claim 37, wherein said plurality of different PPs comprises at least 100 different PPs.

50. The method of claim 37, wherein said plurality of different PPs comprises at least 1000 different PPs.

51. The method of claim 35, wherein PPs comprising said cell sample type of interest PEP comprise at least one cellular PP.

52. The method of claim 51, wherein said cellular PP comprises a regulatory PP.

53. The method of claim 51, wherein said cellular PP comprises a membrane PP.

54. The method of claim 51, wherein said cellular PP comprises a PP from an infectious biologic agent.

55. The method of claim 51, wherein said cellular PP comprises a pathologic or disease related PP.

56. The method of claim 51, wherein said cellular PP comprises a biomarker PP.

57. The method of claim 51, wherein said cellular PP comprises a drug or bioactive compound or candidate or target PP.

58. The method of claim 35, wherein a said PEP is improved in quantitative accuracy, qualitative accuracy, or both, as compared to a PEP compiled from results which are not IRs.

59. The method of claim 35, wherein a said PEP is improved in interpretability as compared to a PEP compiled from results which are not IRs.

60. The method of claim 35, wherein a said PEP is improved in reproducibility as compared to a PEP compiled from results which are not IRs.

61. The method of claim 35, wherein a said PEP is improved in intercomparability as compared to a PEP compiled from results which are not IRs.

62. The method of claim 35, wherein a said PEP is improved in utility as compared to a PEP compiled from results which are not IRs.

63. The method of claim 35, wherein said determining one or more PP improved results (IR) for said cell sample is performed using a microarray assay.

64. The method of claim 35, wherein said determining one or more PP improved results (IR) for said cell sample is performed using a 2D gel electrophoresis assay.

65. The method of claim 35, wherein said determining one or more PP improved results (IR) for said cell sample is performed using at least one affinity binding media, method.

66. The method of claim 35, wherein said determining one or more said PP improved results (IR) for said cell sample is performed using a mass spectroscopy method.

67. The method of claim 35, wherein said determining one or more said PP improved results (IR) for said cell sample is performed using an immunoassay.

68. The method of claim 35, wherein said determining one or more said PP improved results (IR) for said cell sample is performed using an ELISA method.

69. A method for identifying a set of PPs which may be used to identify or characterize a particular cell sample type of interest, comprising
determining improved PP expression results for a plurality of PPs in said particular cell sample type of interest and in at least one reference cell sample type; and
identifying and analyzing PPs which are differentially expressed in the cell sample of interest compared to said reference cell sample type.

70. The method of claim 69, further comprising
selecting at least a subset of said differentially expressed PPs as said set of PPs which may be used to identify or characterize said cell sample type of interest.

71. The method of claim 69, wherein said improved PP expression results are compiled as a PEP for one or more cell samples of the said cell sample type of interest or for one or more cell samples of a specified reference cell sample type of interest, or both; and
said identifying PPs which are differentially expressed comprises comparing protein expression profiles (PEPs) of at least one cell sample type of interest and at least one reference cell sample type of interest.

72. The method of claim 69, wherein one or more regulated PPs are detectably expressed in both the cell sample of interest and said reference cell sample type.

73. The method of claim 69, wherein one or more down regulated PPs is not detectable as being expressed in one of the said cell samples.

74. The method of claim 69, wherein a said cell sample of interest or a said reference cell sample type or both comprises cells from one or more of:
normal cells;
abnormal cells;
untreated cells;
treated cells;
physically treated cells;
chemically treated cells;
drug treated cells;
bioactive compound treated cells;
cells from a psychologically treated individual;
drug candidate treated cells;
toxic compound treated cells;
differentiated cells;
undifferentiated cells;
biological agent infected cells;
virus infected cells;
cells from an individual infected by a pathogenic bacterium;
cells from an individual infected by a eukaryotic microbe;
neoplastic cells;
cancer cells;
diseased cells;
pathological cells;
in vitro cultured cells;
in vitro cultured cells of an immortalized cell line;
in vivo sampled cells;
in vivo sampled cells of a particular tissue;
prokaryotic cells;
eukaryotic cells;
temporally treated cells;
mammalian cells;
mouse cells;
rat cells; and
human cells.

75. The method of claim 69, wherein said cell sample type of interest comprises a plurality of separate different cell sample types.

76. The method of claim 69, wherein said reference cell sample type comprises a plurality of separate different cell sample types.

77. The method of claim 69, wherein said at least one cell sample type of interest or said reference cell sample type or both comprises at least 5 different cell sample types.

78. The method of claim 69, wherein said one or more particular protein (PP) improved results (IR) comprises IRs for a plurality of different PPs.

79. The method of claim 78, wherein said plurality of different PPs comprises at least 3 different PPs.

80. The method of claim 78, wherein said plurality of different PPs comprises at least 10 different PPs.

81. The method of claim 78, wherein said plurality of different PPs comprises at least 100 different PPs.

82. The method of claim 78, wherein said plurality for different PPs comprises at least 1000 different PPs.

83. The method of claim 69, wherein improved protein expression results for a plurality of particular proteins (PPs) are obtained using at least one cellular PP.

84. The method of claim 83, wherein said cellular PP comprises a regulatory protein.

85. The method of claim 83, wherein said cellular PP comprises a membrane PP.

86. The method of claim 83, wherein said cellular PP comprises a PP from an infectious biologic agent.

87. The method of claim 83, wherein said cellular PP comprises a biomarker PP

88. The method of claim 83, wherein said cellular PP comprises a pathologic or disease related PP.

89. The method of claim 83, wherein said cellular PP comprises a drug or bioactive compound candidate or target.

90. The method of claim 71, wherein a said PEP is improved in quantitative accuracy, qualitative accuracy, or both, as compared to a PEP compiled from results which are not IRs.

91. The method of claim 71, wherein a said PEP is improved in interpretability as compared to a PEP compiled from results which are not IRs.

92. The method of claim 71, wherein a said PEP is improved in reproducibility as compared to a PEP compiled from results which are not IRs.

93. The method of claim 71, wherein a said PEP is improved in intercomparability as compared to a PEP compiled from results which are not IRs.

94. The method of claim 71, wherein a said PEP is improved in utility as compared to a PEP compiled from results which are not IRs.

95. The method of claim 69, wherein said determining one or more PP improved results (IR) for said cell sample is performed using a microarray assay.

96. The method of claim 69, wherein said determining one or more PP improved results (IR) for said cell sample is performed using a 2D gel electrophoresis assay.

97. The method of claim 69, wherein said determining one or more PP improved results (IR) for said cell sample is performed using one or more of affinity binding media, methods.

98. The method of claim 69, wherein said determining one or more PP improved results (IR) for said sample is performed using a mass spectroscopy method.

99. The method of claim 69, wherein said determining one or more PP improved results (IR) for said sample is performed using an immunoassay method.

100. The method of claim 69, wherein said determining one or more PP improved results (IR) for said sample is performed using an ELISA method.

101. The method of claim 70, wherein said selecting comprises
identifying from a set of differentially expressed PPs a discrimination set of one or more PPs which can be used to reliably, selectively, and specifically identify individual cell samples of the type of interest and to distinguish said cell samples of interest from the specific reference cell sample type.

102. The method of claim 70, wherein the bases for said selecting comprise the magnitude of the differential expression for a PP.

103. The method of claim 70, wherein the bases for said selecting comprise the consistency of occurrence and direction of the differential expression for a PP.

104. The method of claim 70, wherein the bases for said selecting comprise the magnitude and the consistency of occurrence and direction of the differential expression for a PP.

105. The method of claim 70, wherein said selecting involves application of one or more of the following methods:
a linear discriminant method;
a K-nearest neighbor method;
a neural network method;
a decision tree method;
a partially supervised method;
a class discovery method;
a hierarchical agglomerative clustering method;
a hierarchical divisive clustering method;
a non-hierarchical K-means method;
a self organizing maps and trees method;
a principal component analysis method;
a relationship between clustering and a principal component method;
a protein shaving method;
a clustering in discretised space method;
a graph based clustering method;
a Bayesian model method;
a fuzzy clustering method;
a clustering of proteins and samples method;
a data mining analysis method;
a systems biology analysis method;
an independent component analysis method; and
a direct comparison method.

106. An improved set of cell sample type discrimination particular protein (PP) molecules, comprising
a set of PP molecules which provide specific detection of individual PPs identified by the method of claim 65 which reliably, selectively, and specifically identify individual cell samples of the type of interest, or distinguish said cell sample type of interest from at least one specific reference cell sample type, or both, based on improved PP expression results.

107. The set of discrimination molecules of claim 106, wherein said molecules are labeled or unlabeled or both.

108. The set of discrimination molecules of claim 106, further comprising a set of capture oligonucleotide identifier reagents.

109. The set of discrimination molecules of claim 106, further comprising a set of capture protein or antibody identifier reagents

110. The set of discrimination molecules of claim 108, wherein said identifier reagents comprise an oligonucleotide microarray.

111. The set of discrimination molecules of claim 109, wherein said identifier reagents comprises a protein microarray.

112. The set of discrimination molecules of claim 106, wherein said molecules provide identification of cells of a cancer.

113. The set of discrimination molecules of claim 106, wherein said molecules provide identification of cells infected by an infectious agent.

114. The set of discrimination molecules of claim 106, wherein said molecules provide identification of cells of a developmental state.

115. The set of discrimination molecules of claim 106, wherein said molecules provide identification of cells to a bioactive molecule.

116. The set of discrimination molecules of claim 106, wherein said molecules provide identification of cells exposed to a defined environmental condition.

117. A method for identifying improved sets of PPs for an application utilizing PP expression results, comprising
obtaining improved PP expression results for at least one application pertinent PP; and
selecting a discrimination PP set based on differential PP expression of said PP in at least one application pertinent cell sample type.

118. The method of claim 117, wherein said application pertinent cell type is identified based on a cellular process associated with said application.

119. The method of claim 117, wherein said discrimination PP set is selected utilizing the method of any of claims 69-116.

120. The method of claim 117, wherein said improved PP expression results are obtained utilizing the method of any of claims 1-34.

121. The method of claim 117, wherein said application comprises one or more of:
a data mining analysis;
a systems biology analysis;
a regulatory pathway identification, or analysis, or monitoring, or any two, or all three;
a drug or bioactive compound or biomarker discovery and identification;
a drug or bioactive compound or biomarker validation;
a drug or bio active compound or biomarker development;
a drug or bioactive compound efficacy analysis;
a drug or bioactive compound safety evaluation;
a drug or bioactive compound toxicity evaluation;
a drug or bioactive compound QA/QC evaluation;
a drug or bioactive compound manufacturing monitoring;
a drug or bioactive compound or biomarker related diagnostic test development or use or both;
a particular cell sample of interest related diagnostic test development or use or both;
a disease or pathologic state or both detection or evaluation or both;
a disease or pathologic state or both detection or evaluation or both, before and after administration of a therapeutic treatment;
a disease or pathologic state or both detection or evaluation or both before and after drug administration;
a disease or pathologic state or both detection, monitoring, or prognosis evaluation or any two or all three;
a disease or pathologic state or both detection, monitoring, or prognosis evaluation or any two or all three, before or after drug or other treatment or both;
a drug or bioactive compound commercial product candidate selection;
a drug or bioactive molecule related clinical trial monitoring;
a drug or bioactive compound commercial product candidate market segment identification;
a drug or bioactive compound effectiveness and safety in the treated patient evaluation;
a drug or bioactive compound prescription to the patient selection; and
a monitoring of drug or biomolecule effectiveness or toxicity or both in the treated patient, wherein said monitoring may be long or short term or both.

122. The method of claim 117, further comprising providing a set of particular protein (PP) identifier reagents, wherein members of said set of identifier reagents provide specific detection of corresponding members of a discrimination PP set.

123. The method of claim 117, wherein said set of discrimination molecules comprises at least 3 different discrimination molecules.

124. The method of claim 122, wherein said set of discrimination molecules comprises at least 10 different discrimination molecules.

125. The method of claim 122, wherein said set of discrimination molecules comprises at least 20 different discrimination molecules.

126. A method for producing improved results for an application which directly or indirectly utilizes at least one protein expression profile (PEP) for at least one PP, comprising
utilizing at least one improved PEP directly or indirectly in said application, thereby producing improved application results.

127. The method of claim 126, wherein said PEP is produced according to any of claims 1-34.

128. The method of claim 126 or 127, wherein said PEP is a particular cell sample or cell sample type PEP.

129. The method of any of claims 126-128, wherein said PEP comprises a cell sample PEP comprising a set of one or more regulated PPs which may be used to selectively and specifically identify a particular cell sample type or a particular cell sample type physiological state (PS) of interest or both.

130. The method of any of claims 126-128, wherein said PEP comprises a cell sample PEP comprising a set of one or more regulated PPs which can be used to selectively and specifically identify a particular cell sample type or physiological state of interest.

131. The method of any of claims 126-128, wherein said application comprises application of one or more of:
a linear discriminant method;
a K-nearest neighbor method;
a neural network method;
a decision tree method;
a partially supervised method;
a class discovery method;
a hierarchical agglomerative clustering method;
a hierarchical divisive clustering method;
a non-hierarchical K-means method;
a self organizing maps and trees method;
a principal component analysis method;
a relationship between clustering and a principal component method;
a protein shaving method;
a clustering in discretised space method;
a graph based clustering method;
a Bayesian model method;
a fuzzy clustering method;
a clustering of proteins and samples method;
a data mining analysis method;
a systems biology analysis method;
an independent component analysis method; and
a direct comparison method.

132. The method of any of claims 126-131, wherein said application comprises one or more of:
a data mining analysis;
a systems biology analysis;
a regulatory pathway identification, or analysis, or monitoring, or any two, or all three;
a drug or bioactive compound or biomarker discovery and identification;
a drug or bioactive compound or biomarker validation;
a drug or bio active compound or biomarker development;
a drug or bioactive compound efficacy analysis;
a drug or bioactive compound safety evaluation;
a drug or bioactive compound toxicity evaluation;
a drug or bioactive compound QA/QC evaluation;
a drug or bioactive compound manufacturing monitoring;
a drug or bioactive compound or biomarker related diagnostic test development or use or both;
a particular cell sample of interest related diagnostic test development or use or both;
a disease or pathologic state or both detection or evaluation or both;
a disease or pathologic state or both detection or evaluation or both, before and after administration of a therapeutic treatment;
a disease or pathologic state or both detection or evaluation or both before and after drug administration;
a disease or pathologic state or both detection, monitoring, or prognosis evaluation or any two or all three;
a disease or pathologic state or both detection, monitoring, or prognosis evaluation or any two or all three, before or after drug or other treatment or both;
a drug or bioactive compound commercial product candidate selection;
a drug or bioactive molecule related clinical trial monitoring;
a drug or bioactive compound commercial product candidate market segment identification;
a drug or bioactive compound effectiveness and safety in the treated patient evaluation;
a drug or bioactive compound prescription to the patient selection; and
a monitoring of drug or biomolecule effectiveness or toxicity or both in the treated patient, wherein said monitoring may be long or short term or both.

133. An improved method for identifying regulated PPs which are regulated in response to exposure to a particular treatment, comprising
comparing at least one improved PP expression profile (PEP) incorporating improved results for at least one cell sample exposed to said treatment with at least one improved PEP for at least one reference cell sample, thereby identifying PPs with differential expression in said treated cell sample.

134. The method of claim 133, further comprising cells in said treated cell sample are subjected to said treatment and cells of said reference cell sample are not subjected to said treatment.

135. The method of claim 133, wherein a PEP is provided utilizing the method of any of claims 1-34.

136. The method of claim 133, further comprising utilizing one or more selection processes to identify and rank said regulated PPs based on the magnitude and direction of the change in expression level for said PP in the treated cell sample.

137. The method of claim 136, further comprising
utilizing one or more further selection processes to evaluate the suitability of each of the regulated PPs for the purpose of the comparison; and
interpreting and ranking and arranging the members of said set of regulated PPs and their characteristics in a manner which reflects their suitability of use for the purpose of the said comparison and identification.

138. The method of claim 136 or 137, wherein said selection process involves application of one or more of the following methods:
a linear discriminant method;
a K-nearest neighbor method;
a neural network method;
a decision tree method;
a partially supervised method;
a class discovery method;
a hierarchical agglomerative clustering method;
a hierarchical divisive clustering method;
a non-hierarchical K-means method;
a self organizing maps and trees method;
a principal component analysis method;
a relationship between clustering and a principal component method;
a protein shaving method;
a clustering in discretised space method;
a graph based clustering method;
a Bayesian model method;
a fuzzy clustering method;
a clustering of proteins and samples method;
a data mining analysis method;
a systems biology analysis method;
an independent component analysis method; and
a direct comparison method

139. The method of claim 133, further comprising
exposing at least one of a plurality of matching cell samples to a treatment of interest thereby forming a treated cell sample, while at least one other of said cell sample portions is not exposed to said treatment of interest, and constitutes said reference sample; and
using the method of any of claims 1-34 to produce a PEP for each of said cell samples.

140. The method of any of claims 133-139, wherein the particular treatment comprises one of or a combination of two or more of, the following treatments:
exposure to a compound in a compound screening library;
exposure to a pharmaceutical drug screening hit;
exposure to a pharmaceutical drug lead;
exposure to a pharmaceutical drug;
exposure to a potentially toxic compound;
exposure to a toxic compound;
exposure to an illegal drug;
exposure to protein binding compound;
exposure to an infectious agent;
exposure to a virus;
exposure to a bacterium;
exposure to radiation;
exposure to light;
exposure to ultraviolet light;
exposure to a temperature shift;
exposure to a biological stress condition;
exposure to a psychological stress condition;
exposure to a physical condition;
exposure to a bioactive compound; and
exposure to an environmental condition.

141. The method of any of claims 133-139, wherein one or more regulated PPs are detectably expressed in both said treated cell sample and said reference cell sample type.

142. The method of any of claims 133-139, wherein one or more up regulated PPs is not detectable as being expressed in one of said cell samples.

143. The method of any of claims 133-142, wherein a said cell sample of interest or a said reference cell sample type or both comprises cells from one or more of:
normal cells;
abnormal cells;
untreated cells;
treated cells;
physically treated cells;
chemically treated cells;
drug treated cells;
bioactive compound treated cells;
cells from a psychologically treated individual;
drug candidate treated cells;
toxic compound treated cells;
differentiated cells;
undifferentiated cells;
biological agent infected cells;
virus infected cells;
cells from an individual infected by a pathogenic bacterium;
cells from an individual infected by a eukaryotic microbe;
neoplastic cells;
cancer cells;
diseased cells;
pathological cells;
in vitro cultured cells;
in vitro cultured cells of an immortalized cell line;
in vivo sampled cells;
in vivo sampled cells of a particular tissue;
prokaryotic cells;
eukaryotic cells;
temporally treated cells;
mammalian cells;
mouse cells;
rat cells; and
human cells.

144. The method of any of claims 133-143, wherein said at least one treated cell sample comprises a plurality of separate different cell sample types.

145. The method of any of claims 133-144, wherein said at least one reference cell sample comprises a plurality of separate different cell sample types.

146. The method of any of claims 133-145, wherein said at least one treated cell sample or said at least one reference cell sample or both comprises at least 5 different cell sample types.

147. The method of any of claims 133-146, wherein said one or more PP improved results (IR) comprises IRs for a plurality of different PPs.

148. The method of claim 147, wherein said plurality of different PPs comprises at least 3 different PPs.

149. The method of claim 147, wherein said plurality of different PPs comprises at least 100 different PPs.

150. The method of claim 147, wherein said plurality for different PPs comprises at least 1000 different PPs.

151. The method of any of claims 133-150, wherein improved protein expression results for a plurality of particular proteins (PPs) are obtained using at least one cellular PP.

152. The method of claim 151, wherein said cellular PP comprises .a regulatory PP.

153. The method of claim 151, wherein said cellular PP comprises a membrane PP.

154. The method of claim 151, wherein said cellular PP comprises a protein from an infectious biologic agent.

155. The method of claim 151, wherein said cellular PP comprises a biomarker PP.

156. The method of claim 151, wherein said cellular PP comprises a pathologic or disease related PP.

157. The method of claim 151, wherein said cellular PP comprises a drug or bioactive compound candidate or target.

158. The method of any of claims 133-157, wherein a said PEP is improved in quantitative accuracy, qualitative accuracy, or both, as compared to a PEP compiled from results which are not IRs.

159. The method of any of claims 133-157, wherein a said PEP is improved in interpretability as compared to a PEP compiled from results which are not IRs.

160. The method of any of claims 133-157, wherein a said PEP is improved in reproducibility as compared to a PEP compiled from results which are not IRs.

161. The method of any of claims 133-157, wherein a said PEP is improved in intercomparability as compared to a PEP compiled from results which are not IRs.

162. The method of any of claims 133-161, wherein a said PEP is improved in utility as compared to a PEP compiled from results which are not IRs.

163. The method of any of claims 133-162, wherein said determining one or more PP improved results (IR) for said cell sample is performed using a microarray assay.

164. The method of any of claims 133-162, wherein said determining one or more PP improved results (IR) for said cell sample is performed using a 2D gel electrophoresis method.

165. The method of any of claims 133-162, wherein said determining one or more PP improved results (IR) for said cell sample is performed using at least one affinity binding media method.

166. The method of any of claims 133-162, wherein said determining one or more PP improved results (IR) for said cell sample is performed using an immunoassay.

167. The method of any of claims 133-162, wherein said determining one or more PP improved results (IR) for said cell sample is performed using an ELISA assay.

168. The method of any of claims 133-167, wherein said selecting comprises
identifying from a set of differentially expressed PPs a discrimination set of one or more PPs which can be used to reliably, selectively, and specifically identify individual treated cell samples subjected to a treatment of interest and to distinguish said cell samples of interest from the specific reference cell sample.

169. The method of any of claims 133-168, wherein the bases for said selecting comprise the magnitude of the differential expression for a PP.

170. The method of any of claims 133-169, wherein the bases for said selecting comprise the consistency of occurrence and direction of the differential expression for a particular PP.

171. The method of any of claims 133-170, wherein the bases for said selecting comprise the magnitude and the consistency of occurrence and direction of the differential expression for a particular PP.

172. The method of any of claims 133-171, wherein said selecting involves application of one or more of the following methods:
a linear discriminant method;
a K-nearest neighbor method;
a neural network method;
a decision tree method;
a partially supervised method;
a class discovery method;
a hierarchical agglomerative clustering method;
a hierarchical divisive clustering method;
a non-hierarchical K-means method;
a self organizing maps and trees method;
a principal component analysis method;
a relationship between clustering and a principal component method;
a protein shaving method;
a clustering in discretised space method;
a graph based clustering method;
a Bayesian model method;
a fuzzy clustering method;
a clustering of proteins and samples method;
a data mining analysis method;
a systems biology analysis method;
an independent component analysis method; and
a direct comparison method.

173. The method of any of claims 168-172, further comprising providing a set of PP identifier reagents, wherein members of said set of reagents provide specific detection of corresponding members of said discrimination PP set.

174. The method of claim 173, wherein said set of PP identifier reagents comprises at least 3 different PP identifier reagents.

175. The method of claim 173, wherein said set of PP identifier reagents comprises at least 10 different PP identifier reagents.

176. The method of claim 173, wherein said set of PP identifier reagents comprises at least 20 different PP identifier reagents.

177. The method of claim 173, wherein said set of PP identifier reagents comprises at least 50 different PP identifier reagents.

178. The method of claim 173, wherein said set of PP identifier reagents comprises at least 100 different PP identifier reagents.

179. A method for producing higher order application results which are improved in one or more of qualitative accuracy, quantitative accuracy, interpretability, reproducibility, intercomparability, and utility, relative to prior art produced higher order application results, comprising
using the method of any of claims 1-34 and 126-132, to produce improved results, and
utilizing one or more of said improved results directly or indirectly in a higher order application to produce higher order application results which are improved in one or more of qualitative accuracy, quantitative accuracy, interpretability, reproducibility, intercomparability, and utility, relative to prior art produced higher order application results.

180. The method of claim 179, wherein said higher order application comprises one or more of the following:
a data mining analysis;
a systems biology analysis;
a regulatory pathway identification, or analysis, or monitoring, or any two, or all three;
a drug or bioactive compound or biomarker discovery and identification;
a drug or bioactive compound or biomarker validation;
a drug or bio active compound or biomarker development;
a drug or bioactive compound efficacy analysis;
a drug or bioactive compound safety evaluation;
a drug or bioactive compound toxicity evaluation;
a drug or bioactive compound QA/QC evaluation;
a drug or bioactive compound manufacturing monitoring;
a drug or bioactive compound or biomarker related diagnostic test development or use or both;
a particular cell sample of interest related diagnostic test development or use or both;
a disease or pathologic state or both detection or evaluation or both;
a disease or pathologic state or both detection or evaluation or both, before and after administration of a therapeutic treatment;
a disease or pathologic state or both detection or evaluation or both before and after drug administration;
a disease or pathologic state or both detection, monitoring, or prognosis evaluation or any two or all three;
a disease or pathologic state or both detection, monitoring, or prognosis evaluation or any two or all three, before or after drug or other treatment or both;
a drug or bioactive compound commercial product candidate selection;
a drug or bioactive molecule related clinical trial monitoring;
a drug or bioactive compound commercial product candidate market segment identification;
a drug or bioactive compound effectiveness and safety in the treated patient evaluation;
a drug or bioactive compound prescription to the patient selection; and
a monitoring of drug or biomolecule effectiveness or toxicity or both in the treated patient, wherein said monitoring may be long or short term or both.

181. A method for producing improved information and results concerning the physiological state of cells in a cell sample of a particular cell type of interest, comprising
utilizing one or more particular physiological state PP expression profiles (PS PEPs) to identify the physiological state of different samples of the particular cell type of interest, wherein particular PS PEPs for the particular cell type of interest selectively distinguish a particular physiological state (PS) for said particular cell type of interest,
wherein said PS PEPs are improved by the incorporation of improved protein expression results and wherein said information and results are improved in one or more of qualitative accuracy, quantitative accuracy, interpretability, reproducibility, intercomparability, and utility, relative to prior art produced information and results.

182. The method of claim 181, further comprising
monitoring said physiological state and analyzing the monitoring results to evaluate and determine the physiological state of the particular cell type sample of interest over time and under changing or changed conditions.

183. The method of claim 181 or 182, wherein one or more of the methods of any of claims 1-34 is utilized to produce one or more physiological state PP expression profiles (PS PEPs) for the particular cell type of interest which selectively distinguish a particular physiological state (PS) for said particular cell type of interest.

184. The method of any of claims 181-183, wherein the particular cell type comprises:
a eukaryotic cell type;
a prokaryotic cell type;
a plant cell type;
a bacterial cell type;
a pathogenic bacterial cell type;
a yeast cell type;
a fungal cell type;
a mammalian cell type;
a human cell type;
an in vitro grown cell type;
an immortalized cell line type;
an in vivo grown cell type;
an infectious organism or agent infected cell type;
a virus infected cell type;
a genetically modified cell type;
an in vivo or in vitro cell type used for producing or manufacturing a pharmaceutical agent or protein or small molecule or lipid.

185. The method of any of claims 181-184, wherein the particular physiological state comprises a state selected from the group consisting of:
a cell cycle stage related PS;
a cell growth state related PS;
a cell size related PS;
a differentiated state related PS;
an undifferentiated state related PS;
a toxic state related PS;
a cell age related PS;
an infectious state related PS;
a nutritional state related PS;
a drug or bioactive agent treatment of the cell type related PS;
an environmental state related PS;
a physical treatment of the cell type related PS;
a psychological treatment of the cell type related PS;
a chemical treatment of the cell type related PS; and
a hormone treatment related PS.

186. A method for producing improved clinical trial information and results which are improved in qualitative accuracy, quantitative accuracy, interpretability, reproducibility, intercomparability, or utility, relative to prior art produced such information and results, for the evaluation of one or more or all of the safety, dose, or efficacy of a drug or bioactive agent(BA), comprising
monitoring one or more improved PP expression profiles (PEPs) for drug or BA treated and untreated particular cell types of interest respectively for the appearance of one or more drug treatment desired effects or undesired effects or both in said treated cell types of interest, wherein said improved PEPs incorporate improved protein expression results.

187. The method of claim 186, further comprising analyzing the results of said monitoring to evaluate said safety, dose, and efficacy of the drug or BA treatment of the particular cell types of interest.

188. The method of claim 186 or 187, further comprising utilizing the method of any of claims 1-30 to produce one or more of said particular PEPs.

189. The method of any of claims 186-188, wherein the particular cell types of interest comprise at least one of the following cell types:
eukaryotic;
prokaryotic;
plant;
bacteria;
yeast or fungus;
mammalian;
human;
cell types infected with a biological or other infectious agent;
normal cell types;
abnormal;
pathologic;
untreated;
treated;
psychological treated;
toxic compound treated;
differentiated;
undifferentiated;
neoplastic;
in vitro grown;
in vivo;
diseased; and
pathologic.

190. The method of any of claims 186-189, wherein the PEP comprises a complete PEP for the treated and untreated cell type or types of interest.

191. The method of any of claims 186-189, wherein the PEP comprises a partial PEP specific for a particular treated or untreated cell type or types of interest.

192. The method of any of claims 186-189, wherein the PEP comprises a combination complete and partial PEPs for the treated or untreated cell type or types of interest.

193. The method of any of claims 186-192, wherein the desired or undesired effect comprises
the known desired effects of the drug or BA on the cell types of interest.

194. The method of any of claims 186-192, wherein the desired or undesired effect comprises
the unknown potential desired effects of the drug or BA on the cell types of interest.

195. The method of any of claims 186-192, wherein the desired or undesired effect comprises
the known undesired effects of the drug on the cell types of interest.

196. The method of any of claims 186-192, wherein the desired or undesired effect comprises
the unknown potential undesired effects on the cell types of interest.

197. A method for producing improved information and results concerning the efficacy and toxicity or both or the desired and undesired effects or both, of treatment for a patient being treated with a particular drug or bioactive agent (BA), or with a combination of a plurality of drugs or BAs or both, which is improved in one or more of qualitative accuracy, quantitative accuracy, interpretability, reproducibility, intercomparability, and utility, relative to such prior art produced information and results, comprising
monitoring one or more improved protein expression profiles (PEPs) of patient cell samples for drug or BA treated particular cell types of interest for the appearance of one or more drug treatment desired effects or undesired effects or both in said treated cell types of interest, wherein said improved PEPs incorporate improved protein expression results.

198. The method of claim 197, further comprising analyzing the said monitoring results to determine the effectiveness of the treatment or undesired effects of said treatment or both.

199. The method of claim 197 or 198, further comprising utilizing a method of any of claims 1-34 to produce cell type specific PEPs for the combination of the patient cell types of interest and drug or BA of interest.

200. The method of any of claims 197-199, further comprising comparing at least one PEP for a treated cell sample from said patient with at least one PEP for at least one untreated cell sample.

201. The method of claim 200, wherein said treated cell or said untreated cell sample is from said patient.

202. The method of any of claims 197-201, wherein a PEP comprises a partial PEP specific for a particular treated or untreated cell type or types of interest.

203. The method of any of claims 197-201, wherein the PEP comprises a combination complete and partial PEPs for the treated or untreated cell type or types of interest.

204. The method of any of claims 197-201, wherein the desired or undesired effect comprises
the known desired effects of the drug or BA on the cell types of interest.

205. The method of any of claims 197-201, wherein the desired or undesired effect comprises
the unknown potential desired effects of the drug or BA on the cell types of interest.

206. The method of any of claims 197-201, wherein the desired or undesired effect comprises
the known undesired effects of the drug on the cell types of interest.

207. The method of any of claims 197-201, wherein the desired or undesired effect comprises
the unknown potential undesired effects on the cell types of interest.

208. A method for producing improved patient bioactive agent treatment related health care, comprising
utilizing the method of any of claims 197-207 to determine the effectiveness of the particular drug or bioactive agent (BA) treatment in a patient, and
selecting a drug or BA treatment utilizing the determination of effectiveness information.

209. The method of claim 208, wherein said selecting comprises continuation of treatment with said drug or bioactive agent.

210. The method of claim 208, wherein said selecting comprises an increase in dosage of said drug or bioactive agent.

211. The method of claim 208, wherein said selecting comprises a decrease in dosage of said drug or bioactive agent.

212. The method of claim 208, wherein said selecting comprises termination of treatment with said drug or bioactive agent.

213. The method of claim 208, wherein said selecting comprises administration of an additional drug or bioactive agent.

214. The method of claim 208, wherein said effectiveness information comprises information on the efficacy of said drug or bioactive agent in said patient.

215. The method of claim 208, wherein said effectiveness information comprises information on the safety of said drug or bioactive agent in said patient.

216. The method of claim 208, wherein said effectiveness information comprises tolerance of dosage level information in said patient.

217. The method of any of claims 208-216, wherein said bioactive agent is a food, nutritional supplement, or nutritional compound.

218. A method for producing improved patient bioactive agent treatment related health care, comprising
selecting treatment for a patient based on comparison of at least one improved PEP for said patient, and at least one reference PEP indicative of patient response to said drug or bioactive agent treatment.

219. The method of claim 218, wherein said PEP for a patient is produced by the method of any of claims 1-34.

220. The method of claim 218 or 219, wherein said patient suffers from a disease or condition for which the presence of certain allelic variants is indicative of variation in the effectiveness of treatment with said drug or bioactive agent or indicative of differences in effectiveness of different bioactive agents.

221. The method of any of claims 218-220, wherein the method of any of claims 197-207 is used to determine the effectiveness of the particular drug or bioactive agent (BA) treatment in a patient, and further comprising
utilizing the determination of effectiveness information to select a drug or BA treatment.

222. The method of claim 218, wherein said selecting comprises continuation of treatment with said drug or bioactive agent.

223. The method of claim 218, wherein said selecting comprises an increase in dosage of said drug or bioactive agent.

224. The method of claim 218, wherein said selecting comprises a decrease in dosage of said drug or bioactive agent.

225. The method of claim 218, wherein said selecting comprises termination of treatment with said drug or bioactive agent.

226. The method of claim 218, wherein said selecting comprises administration of an additional drug or bioactive agent.

227. The method of claim 221, wherein said effectiveness information comprises information on the efficacy of said drug or bioactive agent in said patient.

228. The method of claim 221, wherein said effectiveness information comprises information on the safety of said drug or bioactive agent in said patient.

229. The method of claim 221, wherein said effectiveness information comprises tolerance of dosage level information in said patient.

230. An electronic representation of an improved PP expression profile (PEP), comprising
electronic representations of a plurality of improved results obtained by the method of any of claims 1-34.

231. A method for determining improved application results for an application which directly or directly utilizes improved protein expression profile (PEP) information, comprising
entering data describing or derived from said PEP in computer accessible form;
operating on said data with a computer program comprising program steps to calculate said application results.

232. An improved protein array, comprising a set of particular proteins selected by a method of any of claims 69-105 or 117-125, or includes a set of proteins according to any of claims 106-116.

233. The improved protein array of claim 232, wherein said array is a protein microarray chip.

234. A kit comprising at least one improved protein array of claim 232 or 233.

235. The kit of claim 234, further comprising instructions for carrying out an assay using said array, or additional components for carrying out an assay using said array, or both packaged with said array.

236. The kit of claim 235, wherein said additional components comprise one or more of binding solution, wash solution, detection solution, and detection labeling molecule.

237. The kit of claim 236, wherein at least one said solution is provided in dry form suitable for addition of water to form an aqueous solution.

238. A method for normalizing protein expression results, comprising
determining the assay SCR for a differential protein expression assay for at least one cell sample; and
normalizing results of said assay for an assay SCR≠1.

239. The method of claim 238, wherein normalizing is performed for a plurality of particular proteins.
